(19) United States
(12) Patent Application Publication
(10) Pub. No.: US 2025/0127454 A1
(43) Pub. Date: Apr. 24, 2025

(54) GENERATING PATIENT STATES BASED ON PATIENT GOALS

(71) Applicant: Boston Scientific Neuromodulation Corporation, Valencia, CA (US)

(72) Inventors: Dat Thanh Huynh, North Hollywood, CA (US); Matthew Lee McDonald, Glendale, CA (US); Bradley Lawrence Hershey, Carrollton, TX (US); Kristen Marie Lechleiter, Davidson, NC (US); Rex James Woon, San Gabriel, CA (US)

(21) Appl. No.: 18/910,891

(22) Filed: Oct. 9, 2024

Related U.S. Application Data
(60) Provisional application No. 63/544,874, filed on Oct. 19, 2023.

(51) Int. Cl. A61B 5/00; G16H 20/00

(52) U.S. Cl. CPC A61B 5/4836; A61B 5/4824; A61B 5/7475; G16H 20/00

(57) ABSTRACT

Various embodiments of the present subject matter use health-related information to improve the monitoring and treatment provided to remotely managed patient populations by generating patient states to simplify the triaging of patient conditions. In an example, the system can receive from a user via a user interface, criteria related to one or more goals to accomplish while receiving therapy to treat a condition. The system can translate, by at least one hardware processor, the criteria related to the one or more goals to a state-based classification defining a plurality of patient states defined by a plurality of parameters and receive a first set of parameter values. The system can further identify a first patient state from among the plurality of patient states using the first set of parameter values and generate an output including the first patient state.

### PRIORITY CLAIM

This application claims the benefit of U.S. Provisional Application No. 63/544,874, filed on Oct. 19, 2023, which is hereby incorporated by reference in its entirety.

### TECHNICAL FIELD

This document relates generally to medical systems, and more particularly, but not by way of limitation, to systems, devices, machine-readable media, and methods for using healthcare-related data to improve patient monitoring, ease patient triage, and provide treatment options for implanted electrical stimulation for treatment and/or management of pain.

### BACKGROUND

Chronic pain, such as pain present most of the time for a period of six months or longer during the prior year, is a highly pervasive complaint and consistently associated with mental, physical, and/or psychological illnesses. Chronic pain can originate with a trauma, injury or infection, or there can be an ongoing cause of pain. Chronic pain can also present in the absence of any past injury or evidence of body damage. Common chronic pain can include headache, low back pain, cancer pain, arthritis pain, neurogenic pain (pain resulting from damage to the peripheral nerves or to the central nervous system), somatic pain, or psychogenic pain (pain not due to past disease or injury or any visible sign of damage inside or outside the nervous system).

However, chronic pain is far more than the existence of a physical sensation or feeling of pain. Chronic pain is not just a number rating on a scale but can be a life-consuming change to every moment of every day for a pain patient. Chronic pain has a significant impact on a person's quality of life, affecting not only physical health but also emotional and social well-being. Chronic pain can affect a patient's physical limitations, cause emotional and psychological distress, increase social isolation, bring about financial strain, decrease quality of life, and much more.

Neurostimulation, also referred to as neuromodulation, has been proposed as a therapy for a number of conditions, injuries, and causes of chronic pain. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system can include an implantable neurostimulator, also referred to as an implantable pulse generator (IPG), and one or more implantable leads, paddles, or the like each including one or more electrodes. The implantable neurostimulator delivers neurostimulation energy through one or more electrodes placed on or near a target site in the patient's system.

Remote patient monitoring platforms for patients with neurostimulation devices allow clinicians, device representatives, patients, and caregivers to monitor large patient populations. However, it can be difficult and time consuming to go through every patient to determine if they need help. It is desired to improve the management, triaging, and notifications provided to remotely managed patient populations.

### SUMMARY

Various embodiments of the present subject matter categorize a pain patient into one of several patient states to triage the patient for intervention by a clinician, provide patient treatment changes remotely, and/or adjust patient state input sources through adaptation.

Described implementations of the subject matter can include one or more features, alone or in combination as illustrated below by way of example.

Example 1 is a system to monitor a patient, the system comprising: one or more processors; and one or more memory storing instructions, which when executed by the one or more processors, cause the one or more processors to perform operations that: receive, from a user via a user interface, criteria related to one or more goals to accomplish while receiving therapy to treat a condition; translate, by at least one hardware processor, the criteria related to the one or more goals to a state-based classification defining a plurality of patient states defined by a plurality of parameters; receive a first set of parameter values; identify a first patient state from among the plurality of patient states using the first set of parameter values; and generate an output including the first patient state.

In Example 2, the subject matter of Example 1 includes, wherein the plurality of parameters includes at least one physiological parameter.

In Example 3, the subject matter of any of Examples 1-2 includes, wherein the operations to receive the criteria include operations that: receive, from the patient via the user interface, at least one of a patient-supplied ranking of each patient state among the plurality of patient states, a patient-supplied ranking of well-being, or a patient-supplied ranking of each parameter.

In Example 4, the subject matter of any of Examples 1-3 includes, wherein the criteria include at least one of patient-defined criteria, clinician-defined criteria, application-defined criteria, or a combination thereof.

In Example 5, the subject matter of any of Examples 1-4 includes, wherein the instructions perform operations that: receive a second set of parameter values; identify a second patient state from among the plurality of patient states using the second set of parameter values; and generate a comparison state set based on the first set of parameter values and the second set of parameter values.

In Example 6, the subject matter of Example 5 includes, wherein the first set of parameter values and the second set of parameter values comprises at least one of an identical set of parameters, an equivalent set of parameters, or are mapped as a common set of parameters.

In Example 7, the subject matter of any of Examples 1-6 includes, wherein the plurality of patient states includes a representation of overall patient health representing a combination of parameter sets including at least one of: a pain parameter, the pain parameter related to pain of the patient including subjective information provided, via the user interface, by the patient; a medication parameter; an activities of daily living parameter; a mood parameter; a sleep parameter; an alertness parameter; and a mobility parameter.

In Example 8, the subject matter of Example 7 includes, wherein the instructions perform operations that: determine a multi-step pathway between the plurality of patient states based at least in part on the criteria related to the one or more goals to accomplish while receiving the therapy.

In Example 9, the subject matter of Example 8 includes, wherein the instructions perform operations that: identify a time period maintained in the patient state; and calculate a dwell time percentage based on the time period maintained among the plurality of patient states.

In Example 10, the subject matter of Example 9 includes, wherein the instructions perform operations that: map the time period maintained in the among the plurality of patient states to the criteria related to the one or more goals to accomplish while receiving the therapy; generate a recommendations plan for the therapy; and provide the recommendations plan for planning treatment decisions.

In Example 11, the subject matter of Example 10 includes, wherein the instructions perform operations that: identify one or more confounding variables among the plurality of parameters, the one or more confounding variables being identified as problematic to success of the recommendations plan for the therapy.

In Example 12, the subject matter of any of Examples 10-11 includes, wherein the recommendations plan for the therapy is a closed-loop recommendations plan.

In Example 13, the subject matter of any of Examples 10-12 includes, wherein the operations to provide the recommendations plan further comprise instructions to perform operations that: provide the recommendations plan directly to the patient; provide the recommendations plan directly to a clinician; or provide the recommendations plan directly to a stimulator.

In Example 14, the subject matter of Example 13 includes, wherein the instructions perform operations that: detect missing parameter values from the first set of parameter values; receive, from one or more devices operably connected to the patient, adaptive input corresponding to one or more of the missing parameter values; and use the adaptive input to identify the first patient state from among the plurality of patient states.

In Example 15, the subject matter of any of Examples 1-14 includes, the system further comprising: an electro-stimulator configured to generate electrostimulation energy for managing pain of the patient, wherein the electro-stimulator is further configured to deliver at least one of: a device for providing stimulation; a spinal cord stimulation; a brain stimulation; or a peripheral nerve stimulation.

Example 16 is a method, comprising: using at least one medical device to treat a condition by delivering a therapy to a patient; receiving, from a user via a user interface, criteria related to one or more goals to accomplish while receiving therapy to treat a condition; translating, by at least one hardware processor, the criteria related to the one or more goals to a state-based classification defining a plurality of patient states defined by a plurality of parameters; receiving a first set of parameter values; identifying a first patient state from among the plurality of patient states using the first set of parameter values; and generating an output including the first patient state.

In Example 17, the subject matter of Example 16 includes, wherein the plurality of parameters includes at least one physiological parameter.

In Example 18, the subject matter of any of Examples 16-17 includes, receiving, from the patient via the user interface, at least one of a patient-supplied ranking of each patient state among the plurality of patient states, a patient-supplied ranking of well-being, or a patient-supplied ranking of each parameter.

In Example 19, the subject matter of any of Examples 16-18 includes, wherein the criteria include at least one of patient-defined criteria, clinician-defined criteria, application-defined criteria, or a combination thereof.

In Example 20, the subject matter of any of Examples 16-19 includes, receiving a second set of parameter values; identifying a second patient state from among the plurality of patient states using the second set of parameter values; and generating a comparison state set based on the first set of parameter values and the second set of parameter values.

In Example 21, the subject matter of any of Examples 16-20 includes, wherein the plurality of patient states includes a representation of overall patient health representing a combination of parameter sets including at least one of: a pain parameter, the pain parameter related to pain of the patient including subjective information provided, via the user interface, by the patient; a medication parameter; an activities of daily living parameter; a mood parameter; a sleep parameter; an alertness parameter; and a mobility parameter.

In Example 22, the subject matter of any of Examples 16-21 includes, determining a multi-step pathway between the plurality of patient states based at least in part on the criteria related to the one or more goals to accomplish while receiving the therapy.

In Example 23, the subject matter of Example 22 includes, wherein the multi-step pathway between the plurality of patient pain states is a non-sequential pathway from the first patient pain state to a last patient pain state, the last patient pain state being an optimal patient pain state.

In Example 24, the subject matter of any of Examples 16-23 includes, identifying a time period maintained in the patient state; and calculating a dwell time percentage based on the time period maintained among the plurality of patient states.

In Example 25, the subject matter of any of Examples 16-24 includes, mapping the time period maintained in the among the plurality of patient states to the criteria related to the one or more goals to accomplish while receiving the therapy; generating a recommendations plan for the therapy; and providing the recommendations plan for planning treatment decisions.

In Example 26, the subject matter of Example 25 includes, identifying one or more confounding variables among the plurality of parameters, the one or more confounding variables being identified as problematic to success of the recommendations plan for the therapy.

In Example 27, the subject matter of any of Examples 16-26 includes, wherein the recommendations plan for the therapy is a closed-loop recommendations plan.

Example 28 is a machine-storage medium embodying instructions that, when executed by a machine, cause the machine to perform operations comprising: using at least one medical device to treat a condition by delivering a therapy to a patient; receiving, from a user via a user interface, criteria related to one or more goals to accomplish while receiving therapy to treat a condition; translating, by at least one hardware processor, the criteria related to the one or more goals to a state-based classification defining a plurality of patient states defined by a plurality of parameters; receiving a first set of parameter values; identifying a first patient state from among the plurality of patient states using the first set of parameter values; and generating an output including the first patient state.

In Example 29, the subject matter of Example 28 includes, wherein the plurality of parameters includes at least one physiological parameter.

In Example 30, the subject matter of any of Examples 28-29 includes, receiving, from the patient via the user interface, at least one of a patient-supplied ranking of each patient state among the plurality of patient states, a patient-supplied ranking of well-being, or a patient-supplied ranking of each parameter.

In Example 31, the subject matter of Example 30 includes, receiving a second set of parameter values; identifying a second patient state from among the plurality of patient states using the second set of parameter values; and generating a comparison state set based on the first set of parameter values and the second set of parameter values.

In Example 32, the subject matter of any of Examples 28-31 includes, wherein the plurality of patient states includes a representation of overall patient health representing a combination of parameter sets including at least one of: a pain parameter, the pain parameter related to pain of the patient including subjective information provided, via the user interface, by the patient; a medication parameter; an activities of daily living parameter; a mood parameter; a sleep parameter; an alertness parameter; and a mobility parameter.

In Example 33, the subject matter of any of Examples 28-32 includes, determining a multi-step pathway between the plurality of patient states based at least in part on the criteria related to the one or more goals to accomplish while receiving the therapy.

In Example 34, the subject matter of any of Examples 28-33 includes, mapping the time period maintained in the among the plurality of patient states to the criteria related to the one or more goals to accomplish while receiving the therapy; generating a recommendations plan for the therapy; and providing the recommendations plan for planning treatment decisions.

Example 35 is a system for managing pain of a patient, the system comprising: one or more processors; and one or more memory storing instructions, which when executed by the one or more processors, cause the one or more processors to perform operations that: receive, from a user via a user interface, criteria related to one or more goals to accomplish while receiving therapy to treat a condition; translate, by at least one hardware processor, the criteria related to the one or more goals to a state-based classification defining a plurality of patient states defined by a plurality of parameters; receive a first set of parameter values; identify a first patient state from among the plurality of patient states using the first set of parameter values; and generate an output including the first patient state.

Example 36 is at least one machine-readable medium including instructions that, when executed by processing circuitry, cause the processing circuitry to perform operations to implement of any of Examples 1-35.

Example 37 is an apparatus comprising means to implement of any of Examples 1-35.

Example 38 is a system to implement of any of Examples 1-35.

Example 39 is a method to implement of any of Examples 1-35.

This Summary is an overview of some of the teachings of the present application and not intended to be an exclusive or exhaustive treatment of the present subject matter. Further details about the present subject matter are found in the detailed description, figures, and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an embodiment of a monitoring system that can include medical device(s), external system(s), or other healthcare related data source(s) configured for use to collect healthcare-related data for characterization of patient states.

FIG. 2 is a heat map definition of patient states categorized according to different physiological, psychological, and physical metrics used in a patient state monitoring platform, according to one example embodiment.

FIG. 3 illustrates a patient state distribution over a period of time before and after treatment modifications, according to one example embodiment.

FIG. 4 illustrates, by way of example and not limitation, a patient state time distribution for identifying change points in order to trigger alert notifications.

FIG. 5 illustrates, by way of example and not limitation, a block diagram 500 of the patient state monitoring platform for monitoring and testing sequential change point detection in order to determine when alerts should be triggered.

FIG. 6 illustrates, by way of example and not limitation, an embodiment of a patient state monitoring system that alerts users that action is need based on the patient state.

FIGS. 7A and 7B illustrate, by way of example, embodiments of patient state monitoring user interfaces for receiving input and providing output, according to one example.

FIG. 8 illustrates an embodiment of data processing operations being performed on patient state data, in accordance with examples.

FIGS. 9A and 9B illustrate, by way of example and not limitation, processes for identifying multi-step pathways of state transitions without reaching each state.

FIG. 10 illustrates, by way of example and not limitation, a process for identifying a multi-step pathway of state transitions based on patient-ranked preference on preferred pain state and/or based on patient-ranked preference on preferred objectives and/or criteria.

FIG. 11 illustrates, by way of example and not limitation, a patient state change pathway for identifying a different example of a multi-step pathway of state transitions based on patient goals.

FIG. 12A illustrates, by way of example, a block diagram of an embodiment of a system (e.g., a computing system) implementing neurostimulation programming circuitry to cause programming of an implantable electrical neurostimulation device.

FIG. 12B illustrates, by way of example, a block diagram of an embodiment of a system (e.g., a computing system) for performing patient data analysis in connection with closed-loop programming operations.

FIG. 13A illustrates, by way of example, an embodiment of a neurostimulation system.

FIG. 13B illustrates, by way of example and not limitation, a patient system, and examples of devices that may make up components of the patient system.

FIG. 14 illustrates, by way of example and not limitation, using adaptive techniques to determine patient states including adjustments of health-related information.

FIG. 15 illustrates a chart displaying patient states over time through transitions of different cluster solutions, in accordance with one example embodiment.

FIG. 16 illustrates, by way of example and not limitation, a method for computing patient states for remote triaging of chronic pain patients.

FIG. 17 illustrates, by way of example and not limitation, a method for providing a patient state monitoring platform for use with a state-based goal classification system.

FIG. 18 illustrates a method for triggering alerts relating to changes in patient states, in accordance with example embodiments.

FIG. 19 illustrates, by way of example and not limitation, a method for using adaptive input parameters from external data sources to compensate for missing data in patient state monitoring.

FIG. 20 illustrates, by way of example, an embodiment of data interactions among a data analysis computing system and clinician and patient interaction computing devices, for operation or monitoring of a neurostimulation device based on text input analysis.

FIG. 21 illustrates, by way of example, an embodiment of a programming system and data analysis system for use with a neurostimulation system, such as the implantable neurostimulation system of FIG. 24.

FIG. 22 illustrates, by way of example, an embodiment of data interactions among a data analysis computing system and clinician and patient interaction computing devices, for operation or monitoring of a neurostimulation device based on text input analysis.

FIG. 23 illustrates, by way of example, an embodiment of a data processing flow for affecting the neurostimulation treatment of a human patient, based on text and device data processing.

FIG. 24 illustrates, by way of example and not limitation, the neuromodulation system of FIG. 13A implemented in a spinal cord stimulation system or a deep brain stimulation system.

FIG. 25 is a block diagram illustrating a machine in the example form of a computer system, within which a set or sequence of instructions can be executed to cause the machine to perform any one of the methodologies discussed herein, according to an example embodiment.

### DETAILED DESCRIPTION

The following detailed description of the present subject matter refers to the accompanying drawings which show, by way of illustration, specific aspects, and embodiments in which the present subject matter can be practiced. These embodiments are described in sufficient detail to enable those skilled in the art to practice the present subject matter. Other embodiments can be utilized, and structural, logical, and electrical changes can be made without departing from the scope of the present subject matter. References to “an,” “one,” or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

### Overview

Example embodiments of the present disclosure are directed to systems, methods, and machine-readable mediums that include a patient state monitoring and processing platform for patient state triaging, assessment, treatment, and alerts for managing patients with chronic pain. Patient states can include a cluster solution of patient outcomes to remotely simplify a descriptor of a pain patient's health over a period of time or frequency (e.g., on a given day, week, month, quarter, etc.). Chronic pain management can include effective monitoring of patient pain symptoms and physical, functional, and emotional well-being, triggering alert notifications in patient state changes, determining appropriate treatment options (e.g., neuromodulation therapies with an implantable device), and evaluating patient response to pain therapy on a continuous or periodic basis to detect patient state progression and changes in a timely manner.

Existing approaches of chronic pain treatment include conventional in-person visits to a clinic, telehealth appointments, or other scheduled doctor visits, which require person-to-person pain assessments in a clinical setting. Often, a patient will end up needing to provide detailed feedback to a clinician before a treatment issue can be identified and changes can be implemented to a neurostimulation treatment; this can take weeks or even months from onset to triage to treatment implementation.

Traditional approaches for monitoring chronic pain patients include pain assessments of a pain rating, such as a numerical scale of 0 to 10 (where 0 is no pain, and 10 is the worst pain), descriptors of pain intensities, or other measurements of chronic pain. However, such pain ratings can have several shortcomings and/or disadvantages that ultimately cause the patient additional hardships. For example, different patients can have different tolerances to pain, making pain scores subjective and less comparable across patients. Pre-existing solutions for assessing pain, such as the pain rating alone, may not reflect patient physical and functional capacities.

Conventional neurostimulation systems are insensitive to the possibilities that although chronic pain may limit patient functional capacity and cause mobility issues, an improvement in pain sensation (e.g., a lower pain rating) may not always be accompanied by or synchronized with progress in the patient's functional capacities, mobility status, and overall quality of life. For example, a chronic pain patient can report a sizable reduction in pain score yet remains to be bedridden without gaining improvement in their physical or functional capacities. In another example, a chronic pain patient may not report substantial pain reduction, even though they have started walking, sleeping normally, engaging in more activities, changing activities of living, or the like. Such conventional systems fail to provide pain patient support, monitoring, and triaging in everyday life. In addition, existing chronic pain monitoring mechanisms fail to consider or incorporate the vast amount of mental, physical, and functional pain data available to the patient throughout their daily living.

Prior approaches for obtaining patient data from neurostimulation have often attempted to collect subjective data from a patient regarding specific aspects of pain or treatment. Often, prior approaches would use constrained inputs such as visual or numerical scales of pain or discomfort, multiple choice questions and answers, or structured inputs to obtain information from a patient. These inputs often fail to capture the nuance and the significance of historical events, and do not capture the surrounding context that is occurring from a patient. In contrast, the following approaches provide a system which can efficiently and quickly interpret patient parameter input, determine a patient state based on the interpreted patient input, and produce useful alerts for monitoring, diagnosis, treatment, and remediation relevant to neurostimulation device operation.

The capability of a neurostimulation system depends on its post-manufacturing programmability to a great extent. One limiting factor for applications of neurostimulation therapies is that, even if a number of advanced programs can be applied by a neurostimulation device, there is often a delay for implementing new or improved neurostimulation treatments. Such a delay can be due to the infrequency of care provided by a clinician or other medical professional who oversees the treatment, and a lack of clear information regarding the results of the treatment. Various approaches for neurostimulation programming and customization have attempted more dynamic forms of open-loop and closed-loop programing, to allow new neurostimulation parameters or programs to be introduced, deployed, tested, and adjusted by a clinician or the subject patient. Although some neurostimulation devices provide the capability to enable a patient to switch between programs or change the level of a certain stimulation effect, it is often unclear whether such changes (or, which changes) are beneficial to a patient and result in improvement to the patient's medical condition.

Example embodiments of the present disclosure improve upon existing models and overcome such current technical challenges by providing a physiological-based strategy that includes ongoing (e.g., continuous, periodic, timely, etc.) measuring of a patient's patient states progress not in pain relief (e.g., a decrease in pain rating), but measuring progress based on changes in patient states. Patient states can include mental, physiological, and/or physical functioning, such as sleeping, walking, working, connecting with friends, or other behavioral and social activities in daily life, which are grouped into states. Such functional assessments can provide more objective insight into patient physical, emotional, functional, and social capacities, and quality of life (QoL). Such functional assessment insights can be used to evaluate an efficacy of a pain therapy (e.g., neuromodulation therapy) received by the patient, whether the pain therapy needs to be adjusted, when a change in functional pain state should be reported to a clinician, when a change in patient state is worthy of alert, and the like.

Chronic pain can impact the entirety of a patient's life, as a result of that, things like the patient's activity, sleep, mood, eating, etc. can be impacted. Once a stimulator device is implanted in the patient, the treatment options do not stop. It is important for the clinician to understand each individual patient's medical goals, as well as life goals, in order to monitor, customize, adapt, update, and/or change the patient's neurostimulation treatments in an ongoing manner so each patient can move in a direction that satisfies their life and moves the patient toward achieving their goals. For example, two different patients can have the same diagnosis, same neurostimulation device, and drastically different medical and life goals that will enable each patient to live a satisfactory lifestyle. Example embodiments monitor the patient parameters to identify how the therapy afforded by the device can help the patient and clinician identify and meet the needs and goals of the patient, and then, for the patient, understand that they are making progress in the directions that are important for each patient.

For patients with an implantable neuromodulation device for pain control, some patients can experience gradual physiological, functional, or emotional changes, which can go unnoticed for an extended period of time when the patient is outside a clinical setting until it becomes symptomatic and get managed during a clinic visit. Continuous, periodic, or aperiodic monitoring of such patients is desirable to detect pain progression in a timely manner and alert clinicians in changes to patient functional abilities.

Disclosed herein are systems, devices, and methods for monitoring and managing patients with chronic pain using patient states to triage patient pain, monitor and adjust patient goals based on patient states, and provide for monitoring adaptations to patient states. Example embodiments of the present disclosure include systems to remotely monitor patient states and remotely (e.g., outside conventional clinical visits) triage changes in patient states. Such a patient monitoring and pain management platform can monitor patient mobility continuously, periodically, or at any desired time between clinic visits.

According to example embodiments of the present disclosure a patient state monitoring platform is configured to monitor patient states and use the states to identify when a patient has a change in health status. The monitoring platform includes a triaging mechanism that identifies the change in health status and triggers alert notifications to the clinician for on-going treatment updates. The methods and system described herein leverage a multi-criteria decision-making system including a number of patient states (e.g., three states, five states, seven states, etc.) based on different metrics to be used to evaluate different criteria (e.g., objectives) related to chronic pain and provide triaging support mechanisms for treatment modifications in near real-time or real-time. Criteria related to patient pain and objectives in improving the patient's overall health, lifestyle, and/or ability to live in a state of pain can include patient-defined criteria, clinician-defined criteria, application-defined criteria, or a combination thereof. Feedback may include metrics or an efficacy indication reflecting perceived pain, effectiveness of therapies, or other aspects of patient comfort or condition in light of various criteria. Such feedback may be automatically detected from a patient's physiological state (or other states as used throughout the present disclosure), collected from other sensors or devices (not shown), or manually obtained from user input entered in a user interface.

In additional example embodiments, the patient state monitoring platform includes a multi-dimensional state space or multi-dimensional system for identifying, monitoring, updating, and generating notifications user goals related to patient states. The patient states monitoring platform includes using patient states to identify a goal that a user can set, whether the user is the pain patient or a clinician that is tracking the patient's care. Goals can be identified, set, and monitored to keep track of and ensure that the patient is progressing in a positive direction for that specific patient's goals.

In further additional example embodiments, the patient states monitoring platform includes an adaptation system component for using alternative internal and external input sources to identify missing or additional metrics to be used in calculating a patient state. Example embodiments further overcome existing failures in technology by providing using the patient states monitoring platform configured to monitor states and provide recommendations to clinicians on patient outcomes based on patient states.

The patient state monitoring platform and pain management system described herein can be implemented using a combination of hardware and software designed to provide a pain management regimen to increase day-to-day monitoring of chronic pain, to represent patient states, to trigger alerts to clinicians that an action (e.g., a change in programming) should be taken, to increase therapeutic efficacy, to increase patient satisfaction for neurostimulation therapies, to reduce side effects, to increase device longevity, and to help provide other day-to-day pain patient considerations. The present system can be applied in any neurostimulation (neuromodulation) therapies, including but not limited to SCS, DBS, PNS, FES, Vagus Nerve Stimulation (VNS) therapies or other neuromodulation or stimulator therapies. In various examples, instead of providing closed-loop pain therapies, the systems, devices, and methods described herein can be used to monitor the patient and assess patient states when pain either occurs intrinsically or is induced by nerve block procedures or radiofrequency ablation therapies, among others.

### DETAILED EMBODIMENTS

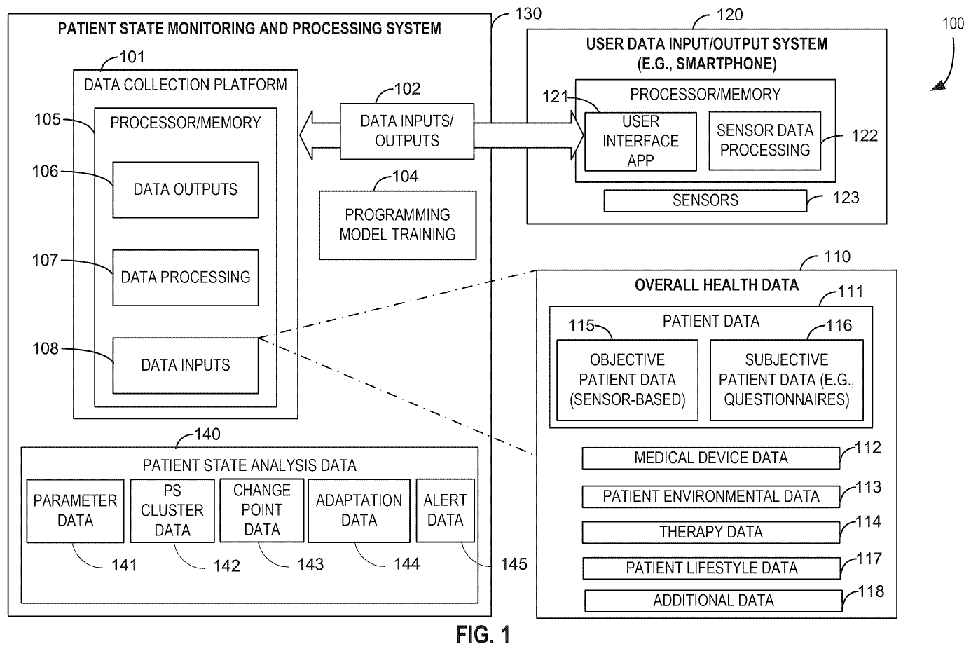

FIG. 1 illustrates a block diagram 100 of a patient state monitoring and processing system 130 that can include medical device(s), external system(s), or other healthcare related data source(s) configured for use to collect healthcare-related data for characterization of patient states. By way of example, an embodiment of patient state monitoring and processing system 130 configured to collect and analyze healthcare-related data in connection with the use of neurostimulation programs and closed-loop neurostimulation programming.

The illustrated patient state monitoring and processing system 130 is configured to include at least one data collection platform 101, which operates to collect and provide data inputs/outputs 102. The data collection platform 101 is configured to process (e.g., filter, extract, transform) the input data to produce patient state analysis data 140, which then can be used for one or more iterations of programming model training 104. The programming model training 104 can be closed-loop programming model, or open-loop system where the action from a controller is independent of the process output (which is the system variable that is being controlled), or other programming models that supply feedback to adjust operation of the system. The data collection platform 101 can also generate output data in the form of recommendations, suggestions, and other information which is relevant to the collection of input data.

The patient state monitoring and processing system 130 may be implemented at one or more server(s) or other systems remotely located from the patient. The patient state monitoring and processing system 130 may use various network protocols to communicate and transfer data through one or more networks which may include the Internet. The patient state monitoring and processing system 130 and data collection platform 101 can include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying training data.

The data inputs 108 can be processed to generate the patient state analysis data 140, which represents a transformed or refined version of data based on actual patient usage, patient feedback (e.g., feedback that indicates what settings have been effective or have not been effective), patient parameter inputs, and the like. The patient state analysis data 140 can include additional data processing including, for example, parameter data 141, patient state cluster solution data 142, change point data 143, adaptation data 144, and alert data 145.

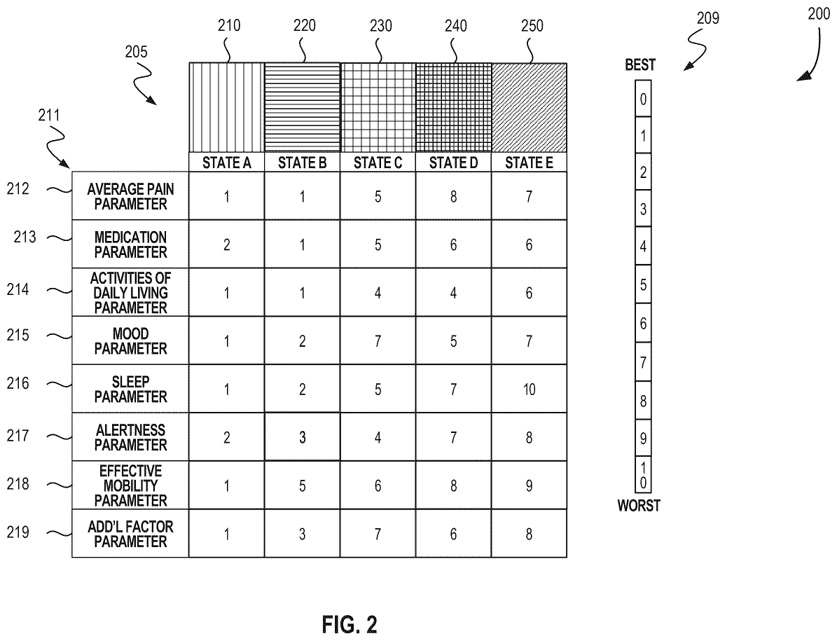

The data inputs 108 may include information obtained from the patient directly and may include various types of overall health data 110 associated with the patient or the treatment. Examples of overall health data 110 may include patient data 111, medical device data 112, patient environmental data 113, therapy data 114, patient lifestyle data 117, additional data 118, or various combinations thereof. The patient data 111 may include objective data 115, such as data collected from physiological sensor(s) and subjective data 116, such as data collected from user-answered question(s) (described in detail in connection with FIG. 2). Additional examples of how objective and subjective data is collected from a patient are discussed in more examples below.

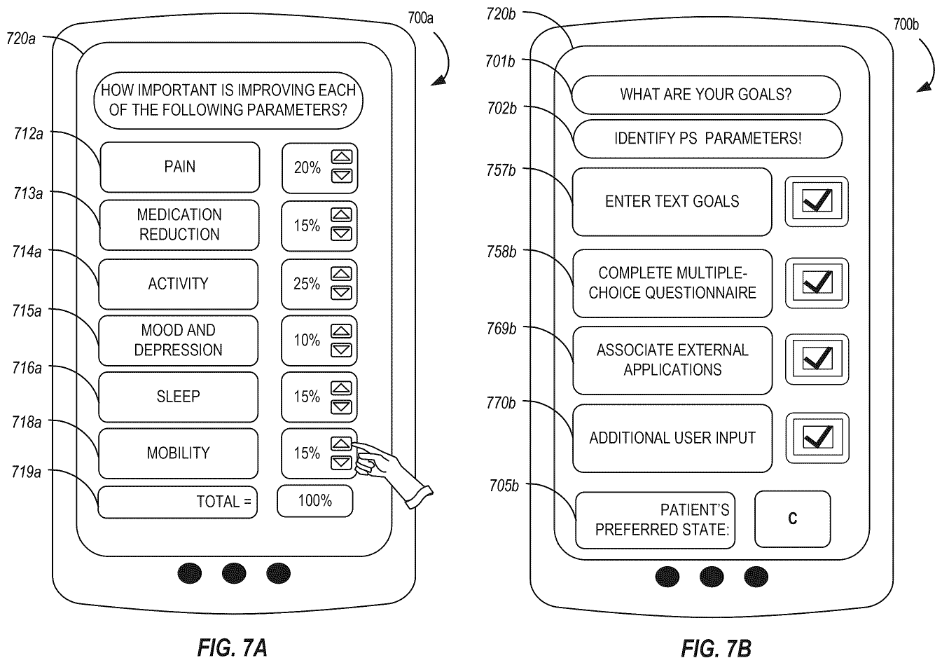

The user data input/output system 120 may be implemented at one or more devices located at or operated by the patient, such as via a smartphone, personal computer, tablet, smart home device, a remote programmer, a programming device, or another compute device or platform capable of collecting input and providing output. The user data input/output system 120 may include at least one processor configured to execute instructions stored in memory (e.g., depicted as processor(s)/memory 105) to provide or generate the data inputs and outputs 102 for receiving or collecting overall health data 110. One such example is a user interface application 121 implemented as a graphical user interface (GUI). Examples of GUIs for data inputs and outputs include smartphone applications that are illustrated in FIGS. 7A and 7B, discussed below. Another example is sensor data processing 122 which can be used to determine the patient physiological state (e.g., amount of activity, duration of sleep) when a particular program is in use.

The user data input/output system 120 may include or interface with sensors 123 such as physiological sensors that can detect patient state information (e.g., activity, sleep), event(s), indicators of device usage, patient compliance with data collection and/or therapy, or various combinations thereof. The user data input/output system 120 may directly or indirectly capture measurements from the sensors 123 or from associated external systems. Examples of external system(s) include remote controls, programmers, phones, tablets, smart watches, personal computers, and the like. The external system may be configured to provide the medical device data 112, the patient environmental data 113, the therapy data 214, patient lifestyle data 117, and/or additional data 118 from an associated device.

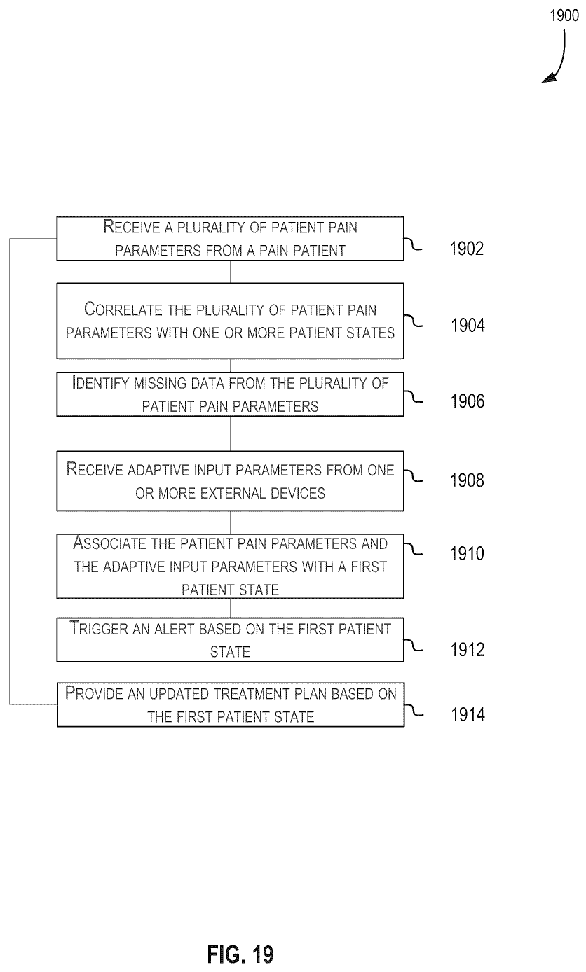

The closed loop programming model training 104 operates to update a programming model by training, re-training, or updating a model (e.g., an artificial intelligence model, such as a neural network) based on the produce patient state analysis data 140. One or multiple instances of a model may be trained to generate programs and program parameters, including with one or more patient-specific models. The closed loop programming model training 104 may consider other aspects of patient-specific or population data such as the overall health data 110, sensor data, and rules and information from a variety of data sources. More details on an example method are described and depicted in connection with FIG. 19.

In some examples, the data collection platform 101 and patient state monitoring and processing system 130 may directly interface with one or more medical device(s), external system(s), or other healthcare-related data source(s) to collect the overall health data 110. One or more of the medical devices, external system(s), and/or other patient-related data source(s) may include technology used by the patient state monitoring and processing system 130 to collect data, and thus may form part of the data collection platform 101. Examples of medical devices include implantable, wearable, and/or operatively interconnected devices. The medical device may be configured to only collect data, to only deliver therapy, or to both collect data and deliver therapy. The medical device may be configured to collect and provide medical device data such as device model, configuration, settings, and the like. Thus, the medical device may provide the patient data 111, the medical device data 112, the environmental data 113, therapy data 114, patient lifestyle data 117, and/or additional data 118, particularly in specialized neurostimulation programming environments.

Other healthcare-related data source(s) may include patient data received via a provider's server that stores patient health records. For example, patients may use a patient portal to access their health records such as test results, doctor notes, prescriptions, and the like. Other healthcare-related data sources may include apps on a patient's smartphone or other computing device, or the data on a server accessed by those apps. By way of example and not limitation, this type of data may include heart rate, blood pressure, weight, and the like collected by the patient in their home. In another example, an app on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like.

The data inputs/outputs 102 may be provided with data transferred via at least one network. The data transfer may use various network protocols to communicate and transfer data through one or more networks which may but does not necessarily include the Internet and/or various wireless networks, which may include short range wireless technology such as Bluetooth. The data may be transferred directly from at least one of the external systems and/or may be transferred directly from at least one of the medical device(s). Further, the external system(s) may be configured to receive data from an associated medical device(s) and/or receive data from other healthcare-related data source(s), and then transfer the data through the network(s) to the data receiving system(s).

FIG. 2 illustrates is a heat map 200 definition of patient states categorized according to different physiological, psychological, and/or physical parameters 211 used in a patient state monitoring platform to measure and analyze overall health of a pain patient into one of five patient states 205, according to one example embodiment. Examples of the system provide for clustering or cluster solutions to be created that define all of the states (e.g., States A-E). In other words, a cluster solution is a definition of the states, which provide cluster definitions (e.g., facts about patients). The cluster definitions provide a set of patient state definitions that provide for a stable state cluster solution. The patient states 205 can include a plethora of patient parameters used to simplify a description of a pain patient's overall health based on the cluster solution. In some example embodiments, patient states can be defined using one or more clustering algorithms, where the selected clustering algorithm has a cluster solution. For example, the heat map 200 defines a cluster solution of States A-E that can be applied to a population of pain patients, and as each patient journeys through varying therapy changes each patient is assigned a state and moves between states of the cluster solution. In alternative example embodiments, the heat map 200 can define one cluster solution for one patient at a specific time or over a specific time period, which can be updated by recalculating the clustering algorithms.

It will be understood by a person having ordinary skill in the art that there can be many cluster solutions to generate different instances of patient states with differing number of clusters and/or cluster definitions. The heat map 200 is one example of a cluster solution for computing patient states (e.g., one definition of patient states) based on a number of parameters 211. For each particular version of states based on each particular cluster solution, the patient state monitoring and processing system 130 utilizes the parameters 211 as data streams for computation of the states. The clustering algorithm is used to find a natural grouping or structure within the same cluster (e.g., within the provided patient data) that are more similar to each other than to objects in other clusters. For example, the clustering algorithm can be a type of unsupervised machine learning algorithm that is used to group or segment a set of data points (e.g., parameter values) into similar subsets or clusters based on their similarity or distance to each other.

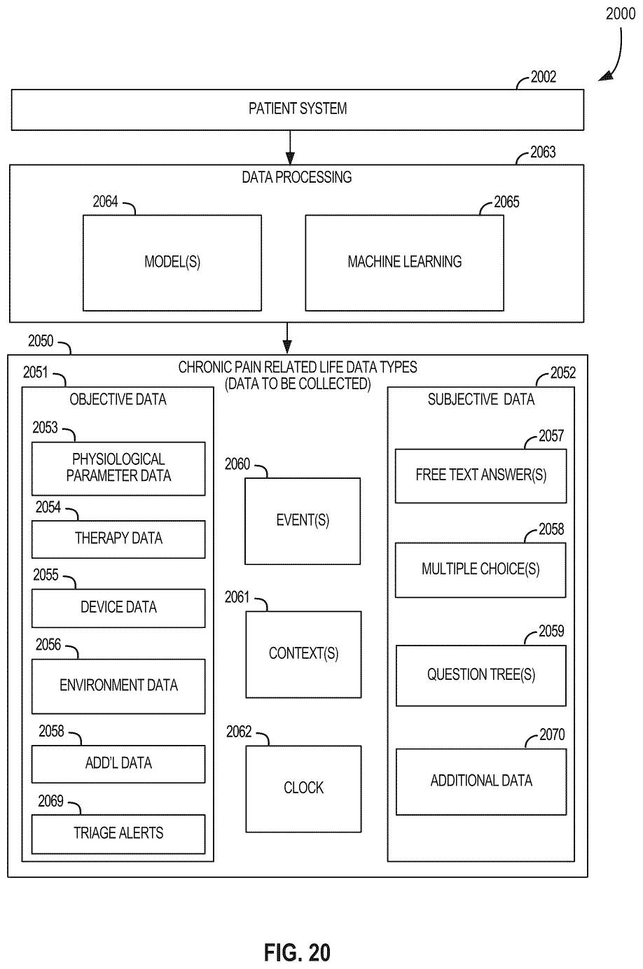

In the example embodiment of FIG. 2, the parameters 211 can be characteristics used alone or in combination to describe and/or define a patient state 205 and can include chronic-pain-related life data, such as healthcare-related data, patient data, medical device data, patient environmental data, therapy data, or combinations thereof. The chronic-pain-related life data can include information obtained from the patient directly (e.g., questionnaires) or indirectly (e.g., adaptive devices, wearable devices) and may include various types of objective and subjective data. Additional examples of how objective and subjective data is collected from a patient is described and depicted in more detail in connection with FIG. 20.

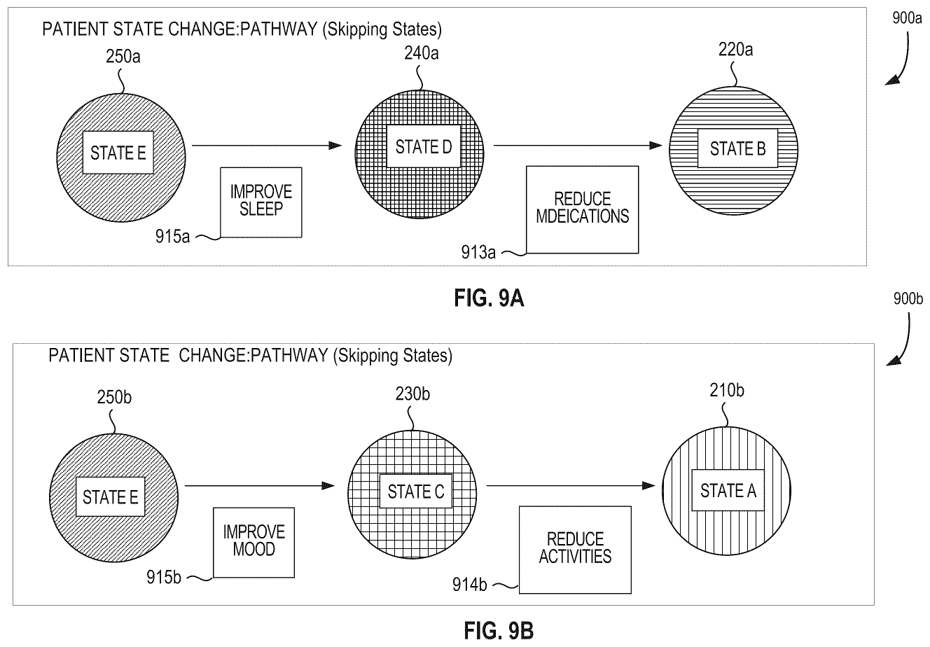

The patient states 205 (e.g., State A 210, State B 220, State C 230, State D 240, State E 250) represent a manageable and straightforward metric (e.g., category, label, etc.) to simplify the overall health of the patient into a single value (e.g., a state category, an overall health score class, etc.). In the example embodiment of FIG. 2, the patient states 205 include a first state, State A 210, a second state, State B 220, a third state, State C 230, a fourth state, State D 240, and a fifth state, State E 250 (where each state is represented by varying pattern fills (e.g., gradients, colors, lines, etc.) that are used throughout the figures to differentiate between states). The heat map 200 illustrates five levels of overall health of the patient, where State A 210 is a higher state of overall health than State B 220, which is a higher state of overall health than State C 230, which is a higher state of overall health than State D 240, which is a higher state of overall health than State E 250 (being the lowest state of overall health). In some examples, the system has found five clusters that can be ordered ordinally based on physiologic metrics and other parameters to form a global optimal order. For example, State A is the highest (e.g., best) state in the global optimal order and State E is the lowest (e.g., worst) state in the global optimal order. While the global optimal order exists, how patients traverse through the states (or through the order) can be different. For example, a patient's preferred state may not align with the global optimal order; in other words, a patient may have a patient optimal order and/or a patient optimal state preference that is different from the global optimal order, or a clinician optimal order or an application optimal order (e.g., based on algorithmic calculations). In some pain cases, a patient optimal order, or a patient preferred optimum state, may or may not be obtainable. In other pain cases, the global optimal order may or may not be obtainable. Example embodiment of the system provide for pathways between states based on the known global optimal order and the patient optimal order are described and depicted in connection with FIGS. 9A, 9B, 10, and 11.

As the life and overall health of a chronic pain patient is affected by more than just the physical sensation of pain, example embodiments of the present disclosure provide a patient state monitoring system that considers health-related data and parameters 211 connected to a patient's totality of affected life. The parameters 211 (e.g., metrics of daily life) can include a pain parameter 212, a medication parameter 213, an activities of daily living parameter 214, a mood parameter 215, a sleep parameter 216, an alertness parameter 217, an effective mobility parameter 218, and/or additional factor parameters 219, where each parameter is utilized to create groupings of states that represent a patient state 205. The patient states 205 can include any number of states to represent an objective and quantitative measure of a pain patient's overall quality of life. For example, in the heat map 200, there are five patient states (e.g., States A-E) generated to represent different quality of life categories to place a pain patient in based on values of the parameters 211, although in varying embodiments or for varying patients, a different number of states (e.g., three states, seven states, nine states, etc.) can define the heat map.

For example, patient chronic pain can make it difficult to perform daily tasks and activities, such as walking, standing, sitting, showering, dressing, using the restroom, exercising, and the like; it can also lead to decreased mobility and range of motion. Chronic pain can cause emotional and psychological distress, such as depression, anxiety, stress, frustration, feelings of failure, and the like; it can also affect a person's self-esteem and self-confidence. Chronic pain can interfere with sleep, causing insomnia, restless sleep, interruptions to sleep, nightmares, and the like, leading to fatigue, brain fog, exhaustion, etc. Further, pain can make it difficult to leave the patient's home, difficult to socialize, hard to engage in social activities, leading to social isolation, loneliness, loss of friends, and feelings of helplessness. Chronic pain can lead to decreased work productivity, loss of ability to work, increased medical expenses, and the like, resulting in financial strain. In other words, chronic pain can overall decrease a patient's quality of life making it difficult to enjoy hobbies and activities that were once pleasurable.

Each of the parameters 211 can be a category of parameters that include a series of one or more patient responses to questions provided by the patient state monitoring and processing system 130. For example, a questionnaire providing lifestyle questions that a patient can respond to using a plethora of response types, such as free text responses, percentage input responses, visual representation answers, multiple-choice answers, and/or additional patient input to respond to the provided questions.

For example, questions provided to the patient for response to medication parameters 213 can include: “Did you need to take prescribed opioid, pain, sleep, muscle relaxant medication for your pain today,” “Did you need to take over-the-counter medication today,” “Did you need to take herbal supplements today,” “Did your medication help improve your pain level or overall pain health,” or the like.

For example, questions provided to the patient for response to activity of daily life 214 parameters can include: “What type of activities did you do today,” “Did your pain level interfere with your activities,” “What, if any, pre-pain hobbies did you do today that you are not normally able to do,” “What were the levels of your activities today,” “How many times did you leave your home today,” “Were you able to work,” “Did you have any social interaction today,” “Did you have any doctor or practitioner appointments today,” or the like.

For example, questions provided to the patient for response related to mood parameters 215 can include: “Rate your mood,” “How is your energy level,” “Are there life sources of stress unrelated to chronic pain,” “Have you been irritable,” “What emotions are you feeling today,” or the like.

For example, questions provided to the patient for response related to sleep parameters 216 can include: “Please rate the quality of your sleep,” “How many hours did you sleep in the last day,” “How many times did you wake up last night,” “How long did it take you to fall asleep,” or the like.

For example, questions provided to the patient for response to alertness parameters 217 can include: “How would you rate your attentiveness level today,” “How would you rate your alertness level today,” “How would you rate your motivation today,” “Did you feel excessive sleepiness during the day,” “How would you rate your concentration during activities,” or the like.

For example, questions provided to the patient for response to effective mobility parameters 218 can include: “Were your movements hindered today,” “How would you assess your mobility levels,” “How independent were you today,” “Did you need excessive help with daily tasks today,” “Did you feel any weakness,” “Did you use your mobility aides today,” “Did you feel safe in your movements today,” “Did you encounter anything you were unable to do due to mobility problems,” or the like.

For example, questions provided to the patient for response to additional factor parameters 219 can include: “What type of symptoms did you experience today,” “What do you wish you could have done differently or additionally today,” “What wearable devices did you wear or use today,” “Are there any other proposed questions and answers you would like to use,” or other patient lifestyle, health-related, or other such patient input that can be used as a parameter in determining a patient state.

For one or more of the questions, a severity score scale 209, such as a 0 to 10 rating scale, where 0 is “best” and 10 is “worst” in response to severity of the response. For example, the severity score scale 209 can be an alpha-numeric score rating scale. For questions that require freeform responses, multiple choice responses, or other non-numerical responses, the patient state monitoring and processing system 130 can analyze the patient responses and automatically or manually provide a representative numeric value for the answer to each question. The numeric values for each question in each parameter are tabulated to generate a single numerical value for each parameter 212-219. In additional example embodiments, the heat map 200 can be generated using colors, where green is “best,” and red is “worst” on the severity score scale 209. This version of the severity score would including varying ranges of colors to display in place of number values.

Returning to the example embodiment of FIG. 2, the patient states 205 (e.g., States A-E) generated from a clustering algorithm simplify the plethora of parameters into distinct, discrete categories based on certain criteria or characteristics. The overall health of a pain patient is divided into states in order to understand the patient's pain behavior at different points in time or under different inputs. The example embodiment of FIG. 2 defines the five states 205 (e.g., States A-E) based on the vales of the variables or execution of specific functions to create the parameters 211. In the exemplary heat map 200 (e.g., for a single patient during a period of time), State A 210 can be defined as having severity score scale 209 values of two or less; State B 220 can be defined as having severity score scale 209 values of five or less; State C 230 can be defined as having severity score scale 209 values of seven or less; State D 240 can be defined as having severity score scale 209 values of eight or less; and State E 250 can be defined as having severity score scale 209 values of ten or less.

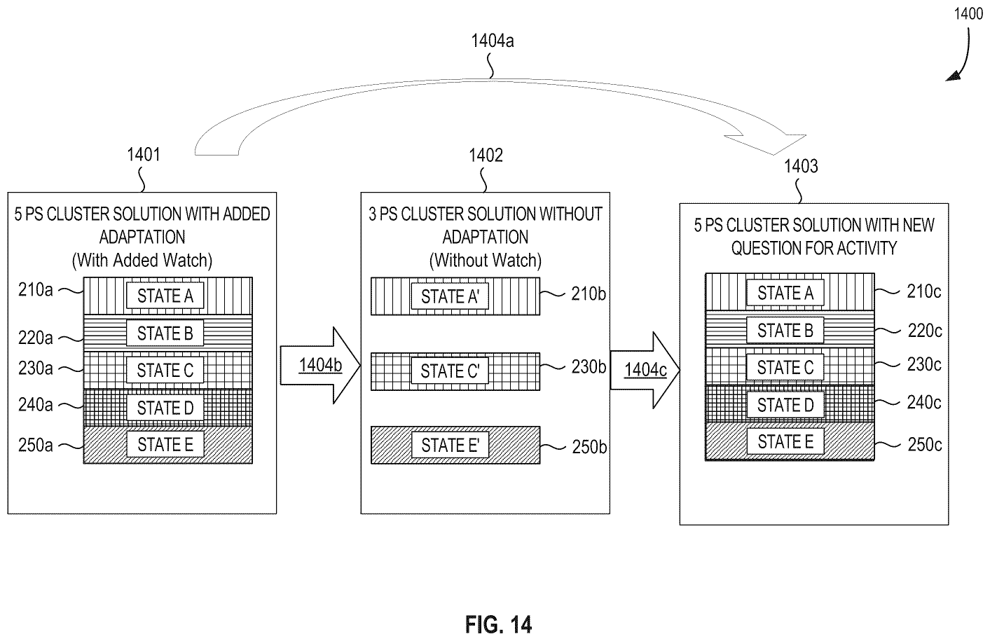

According to some examples, patient input of values (e.g., severity score scale 209 values 0 through 10) for each parameter 211 can provide two distinct pieces of information based on the same underlying values. For example, the same underlying values are available at two time points (e.g., periods of time) or for the same underlying parameters (e.g., sleep parameter, alertness parameter, mobility parameter, etc.) there are different values at two time points. In further examples, patient input (e.g., values) may be missing and adaptive technology/techniques may be used to provide for missing values (described and depicted in connection with FIGS. 14, 15, and 19).

By identifying distinct patient states and the transitions between them, a user can gain insight into the behavior or overall health of the patient, develop strategies for optimizing performance of a neurostimulation device, triage patient problems in a timely and efficient manner, trigger alerts to users, and/or address any treatment problems that arise on a daily basis. According to example embodiments of the present disclosure, the user can be a patient, clinician, doctor, caregiver, device representative, or the like.

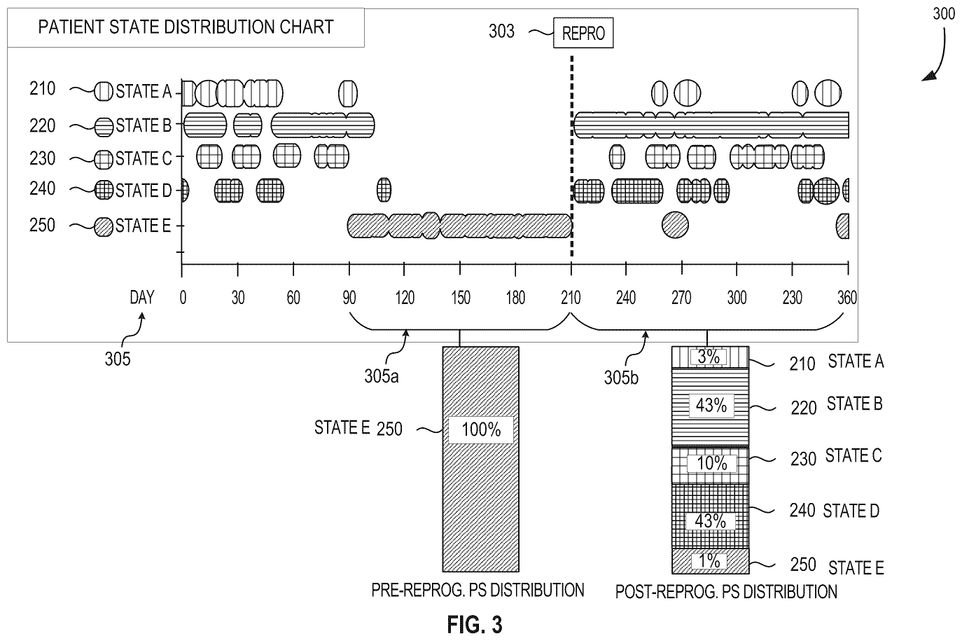

FIG. 3 illustrates a patient state distribution chart 300 over a period of time 305 before and after treatment modifications, according to one example embodiment. The patient state distribution chart 300 (e.g., line graph) is used to display patient state data over time, where the horizontal x-axis represents time 305 (e.g., one year in thirty-day increments) and the vertical y-axis represents the patient state being measured (e.g., States A-E). Each data point on the time graph is plotted at specific points in time, with each point representing the value (e.g., A-E) of the variable (e.g., patient state) being measured at that particular time.

In the example embodiment of FIG. 3, the patient state distribution chart 300 identifies a first time period 305a that includes a pre-reprograming patient state distribution showing a 120-day period of time where the patient's overall health score was identified in State E 250. At day 210, after three full months at the lowest health score defined for that patient, the patient underwent a neurostimulation reprogramming 303. After the reprogramming 303, the patient state distribution chart 300 identifies a second time period 305b that includes a post-reprogramming patient state distribution showing a 150-day period of time where the patient's overall health score oscillates between all five states for varying amounts of time. For example, after the reprogramming 303, the patient spent 1% of the 150-day period of time in State E 250, 43% of the time in State D 240, 10% of the time in State C 230, 43% of the time in State B 220, and 3% of the time in State A 210.

Dwell time can include a percent of time spent in each patient state over a prescribed period of time (e.g., time period). For example, there can be a first time period and a second time period that the system calculates over, and there can be a dwell time percentage for a particular state (e.g., a first state) in each of those time periods. The dwell time percentage provides a measure of how much time is spent in a particular state relative to the total time. For example, in order to determine a dwell time, once the system has a definition of each of the patient states, the system monitors the patient over a period of time (e.g., 10 days) and determines over that period of time (e.g., during that 10-day period of time), what percent of time was the patient in each of the defined states. In some examples, dwell time

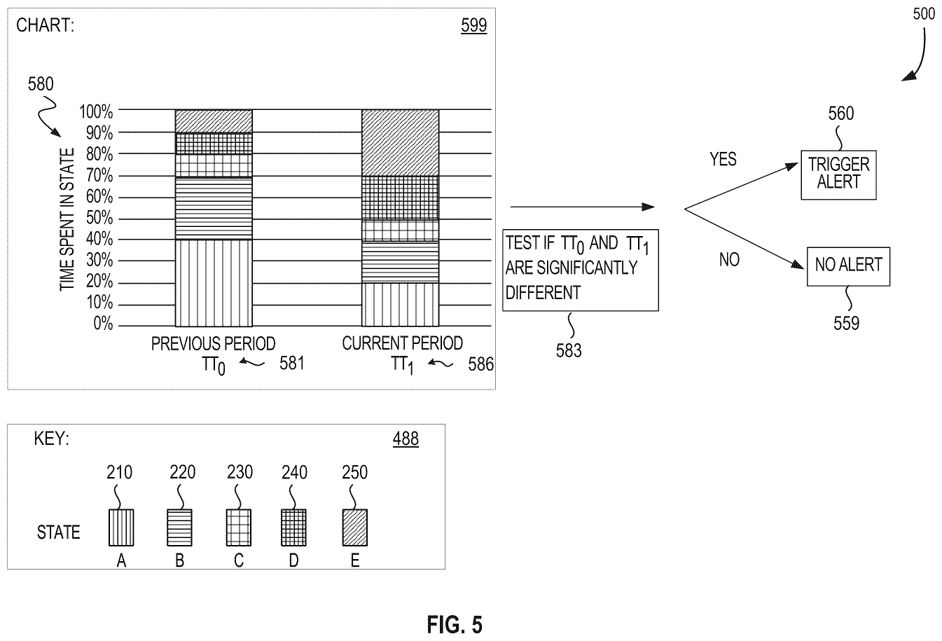

Dwell time, in the context of distributions, can refer to the length of time a patient stays in a particular state before transitioning to another state. In a statistical distribution, it could represent the probability distribution of the time that the patient spends in a particular state. This concept is often used in stochastic processes and Markov models (e.g., where systems transition between states randomly with certain probabilities). The dwell time can provide important information about the patient's pain including data related to the behavior of the treatments for the pain patient, the behavior of the patient's pain, the behavior of the stimulation device, the behavior of the parameters or metrics used in determining patient states, and the like. For example, such as how long on average the patient stays in a particular patient state, or the probability that the patient stays in that patient state for a certain length of time. Dwell time is further described and depicted in connection with FIGS. 5, 6, and 10.

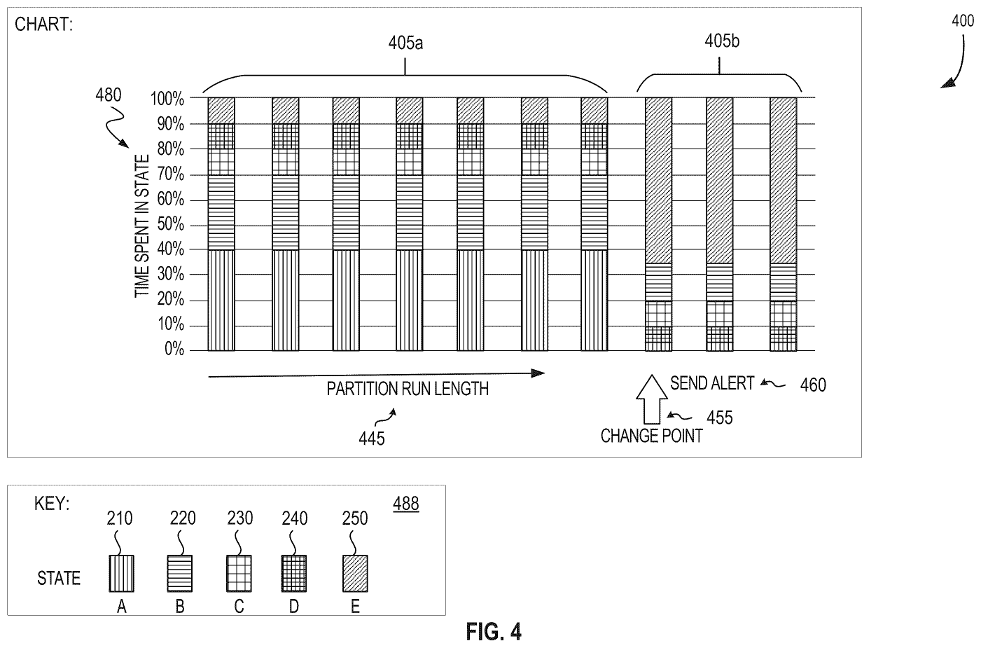

FIG. 4 illustrates a patient state time distribution chart 400 for identifying change points in order to trigger alert notifications. The chart 400 displays each of the five defined patient states (e.g., States A-E), where each portion of the bar graph corresponds with a different fill pattern according to key 488.

The patient state distribution chart 400 (e.g., bar graph) is used to display time spent in patient states over partition run length, where the horizontal x-axis represents partition run length 445 and the vertical y-axis represents the time the patient spends in each patient state 480 being measured (e.g., States A-E). The chart 400 further identifies when a change occurs, enabling the patient state monitoring and processing system 130 to send an alert 460 to the user (e.g., clinician, patient, etc.).

For example, the chart 400 displays two groupings of time periods 405a, 405b, where the first time period 405a shows a patient state distribution that is identified as acceptable for and/or by the user (or system) and where the second time period 405b shows a patient state distribution that is identified as unacceptable for and/or by the user (or system). In the first time period 405a, the patient state distribution shows that the patient is categorized in State A 210 for 40% of the time, the patient is categorized in State B 220 for 30% of the time, and the patient is categorized in States C 230, D 240, and E 250 for 10% of the time, respectively. Then, a change point 455 is detected at the start of the second time period 405b, where the patient state distribution identifies the patient as being in an overall worse state of health for a designated extended period of time. For example, in the second time period 405b, the patient state distribution shows that the patient is categorized in State A 210 for 3% of the time, the patient is categorized in State D 240 for 7% of the time, State C 230 for 10% of the time, State B 220 for 15% of the time, and State E 250 for 65% of the time.

The patient state monitoring and processing system 130, or other component of the neurostimulation system described throughout, detects a change in patient state distribution (e.g., change point 455). The change point 455 can be a structural break that refers to a point in time or in sequence of data where there is a notable change or shift in the underlying statistical properties of the data. For example, the change point can be used to identify the point(s) where there is a change in the mean, distribution, variance, or other properties of the patient state data. The change point 455 is utilized by the system to detect shifts in trends, sudden jumps or drops in values, and other structural changes in the patient state data that may identify a need for patient triage, patient treatment changes, user alerts, or the like.

In some example embodiments, the change point 455 can be analyzed to identify the locations and magnitudes of change points in the data, as well as estimating the characteristics of the data before and after the change point 455. The change point analysis can help to reveal underlying patient state and/or parameter patterns that may be of interest to the user. Example embodiments of the change point analysis can be identified using a variety of statistical methods, such as Bayesian modeling, non-parametric techniques, hypothesis testing, or the like. The change point analysis can further be used to identify acceptable versus unacceptable changes in patient state distribution, patient state, and/or parameters associated with patient states.

Additional example embodiments of the present disclosure include the patient state monitoring and processing system 130 further includes the change point 455 being automatically detected based on a user setting, a prespecified threshold for change, a system-determined anomaly, a patient-identified difference, or other bases for identifying a change in distribution. For example, the patient or clinician can specify to the system what kind of change in data or patient state distribution qualifies as being significant in order to trigger an alert 460. For some patients, only downward trends in patient states may be considered relevant to trigger an alert. For other patients, stagnation in a state may be considered most important for triggering an alert. For still other patients, any change (e.g., positive, negative, or unchanged) in state, change in distribution, and/or any time period may be considered relevant to trigger an alert.

In additional example embodiments, detecting change points and/or approximating a stationary distribution (π) can be performed according to other methods. For example, assuming a patient is in stable health and therapy is not changing, the state dwell time for a sufficiently long period of time approximates the stationary distribution (π) of a discrete-time Markov chain. A discrete-time Markov chain can be a mathematical model for describing a sequence of events or states that occur over time, where the probability of transitioning to a new state only depends on the current state (e.g., current period π₁ 586) and not on any past states (e.g., previous period π₀ 581, or other previous periods). Additional mathematical models can be used to determine dwell time according to the present disclosure.

However, patients are not always in stable health; so additional example embodiments can include dividing a sequence of observed states x₁, x₂, x₃, . . . x_{T} (where x denotes State x) into non-overlapping partitions, where each partition has its own stationary distribution (πᵢ). Delineations can be defined between each partition as a change point. For example, the change point can be detected by prespecifying a threshold for change according to statistical modelling and regression structures. Additional example embodiments can detect the change point by jointly estimating a run length and the stationary distribution using an online change point detection algorithm, such as Bayesian online changepoint detection. Further additional example embodiments using such algorithms can include transmitting an alert in response to a change point detection.

In some example embodiments, when the patient state distribution is identified as unacceptable, the patient state monitoring and processing system 130 automatically triggers a triage alert 460 to the user. The alert 460 is described and depicted in more detail in connection with FIGS. 5, 6, and 8.

FIG. 5 illustrates a block diagram 500 of the patient state monitoring platform for monitoring and testing sequential change point detection in order to determine when alerts should be triggered according to one example embodiment. The chart 599 displays each of the five defined patient states (e.g., States A-E, States A-Z, etc.), where each portion of the bar graph corresponds with a different fill pattern according to key 488.

As depicted in chart 599, the patient state monitoring and processing system 130 monitors, on a continuous, periodic, or aperiodic basis, the time a patient spends in each specific patient state. For example, the chart 599 depicts two time periods, a first time period, previous period π₀ 581, and a second time period, current period π₁ 586. The system tests 583 to determine if the previous period π₀ 581 and the current period π₁ 586 are significantly different. If the system determines a significant difference in distribution, an alert is triggered 560; if no significant difference is determined, no alert is triggered 559. In varying embodiments, the determination of “significance” can be defined on a per-user basis, per-diagnosis basis, per-representative basis, or other determinations.

For example, in the example embodiment of FIG. 5, the system evaluates the divergence of the stationary distribution over time. State dwell time can be defined by the percentage of time in a state over a fixed period of time. According to the example embodiment in the chart 599, the dwell time is represented by a bar where the bar is divided into each percentage for each state. The patient state monitoring and processing system 130 can be utilized as a feedback system, where the state dwell time can affect the system's response to changes in the input parameters, the ways and timing of patient triage, the system's prompting user alerts, the system's identification of specific problematic parameters, the system's capacity to impute data or utilize data adaptations, the system's ability to identify underlying causes (e.g., what has gone wrong with the patient) based on the input parameters, and/or the system's providing effective treatment plans to stabilize the patient state.

For example, the patient state monitoring and processing system 130 can include comparing observed dwell time in period t (with distribution πₜ) versus the dwell time in time period (t−1) (with distribution πₜ₋₁). Alternative methods for detecting change points can include using a chi-square test to determine whether there is a significant association between two categorical variables (e.g., between the state dwell times). Using the chi-square test to determine association of dwell times, the test compares the observed frequency of occurrence of the dwell times with an expected frequency of occurrence to calculate the chi-square statistic. Distributions dominated by one or two patient states would require smaller samples to assess for significant differences.

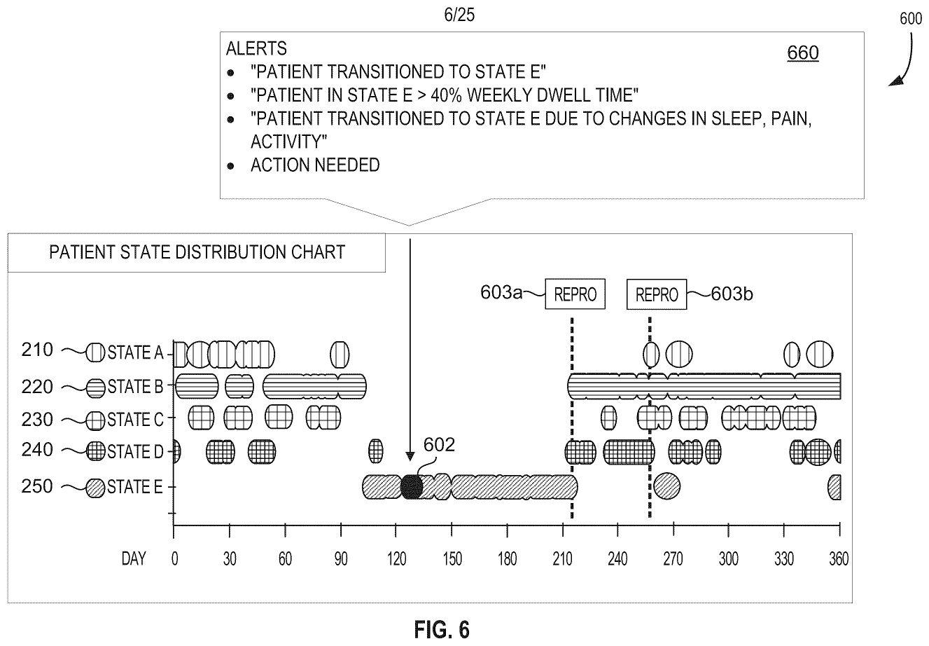

FIG. 6 illustrates, by way of example and not limitation, a patient state distribution chart 600 that alerts users that action is need based on the patient state. As may be clearly visualized, the patient state distribution chart 600 as depicted shares similar features and components as the patient state distribution chart 300 as illustrated and described in connection with FIG. 3. For brevity, only specific elements are detailed and described with reference to FIG. 6.

While using generic monitoring platforms, doctors can examine patient outcomes, but it is a time-intensive process to review hundreds of patients with numerous diagnoses, statuses, symptoms, and changes. The patient state monitoring and processing system 130 can process patient parameter input and generate the patient state distribution chart 600 for providing patient state data plotted over time (e.g., 360 days) to help clinicians monitor, triage, and treat patient symptoms based on the representative pain patient's health state (e.g., State A-E), which is derived from multiple parameters, outcomes, and timely data. The monitoring system can further utilize the patient state distribution chart 600 to generate an alert to one or more users (e.g., clinician and patient) that an action (e.g., treatment change) is advised based on the patient state.

For example, one or more alerts 660 can be generated and transmitted to a user at time 602 (e.g., at day 130) in response to a change in patient state. The one or more alerts 660 can include information identifying the patient state change that has occurred (e.g., “patient transitioned to State E”), a dwell time amount or percentage in the identified state (e.g., “patient in State E for greater than 40% weekly dwell time”), and/or the system-derived (or patient-provided) identification of one or more underlying causes of the patient state change. Additional example embodiments can identify underlying trends in patient state dwell times over a period of time and present a set of the changes that occurred (e.g., patient parameter inputs, patient goal changes, etc.) to the user in an alert. Alerts can further be triggered based on reprogramming sessions 603a and 603b to notify the clinician that the patient and device representative have changed patient programming.

One goal of the system is to provide users with alerts so evaluation and intervention can be implemented as soon as possible. The alerts 660 provide for simplified notifications of state changes without presenting overwhelming amounts of data. For example, there can be a vast amount of data involved in monitoring patient health, so there is the possibility of data overload. This invention allows for triggering alerts of relevant changes so the clinician is not just made aware of every change in state but made aware of changes that should result in modifications to the patient's treatment plan.

Additional example embodiments of the alerts 660 include one or more algorithms for determining when a patient moves or transitions into a “bad” state or moves into a distribution of states that is different than previous distributions that can be used by the patient state monitoring and processing system 130 to determine when to generate an alert. The system can provide for setting of thresholds of sensitivity of changes for each individual patient and generate alerts based on the sensitivity thresholds. For example, the system can include user-defined thresholds on patient state changes, state dwell times, change in patient state variability, patient state event detection, or user-customized alert settings. The user-defined alert settings can also be different based on the end-user viewing the alerts. For example, the clinician may be interested in specific alerts that are different than alerts generated for the patient or the device representative.

In additional example embodiments of the alerts 660, in response to generating an alert, the patient state monitoring and processing system 130 can suggest or take an action based on the alert. For example, in response to automatic detection of change points that cause an alert, the system can provide patient data statistics to the user at a more granular focus than the patient state. Additional automatic actions based on the alert can include prior suggested patient programming settings that may be beneficial to implement while waiting for the clinician, or other generalized or specific guidance.

FIGS. 7A to 7B depict data input user interfaces 720a, 720b (e.g., a smartphone app graphical user interface), which provide output on a user device 700a, 700b (e.g., a smartphone, application, computer, remote, etc.). In various examples, a patient's phone, computer, tablet, or programming remote control (or multiple of these devices) can provide the illustrated user interfaces.

FIG. 7A depicts the data input user interface 720a as including a number of patient inputs that the patient can enter and update over a period of time (e.g., hourly, daily, weekly, seasonally, etc.) regarding patient state parameters 211. For example, the patient can provide a numerical percentage on the importance of improving different parameters. Other user interface examples can include a selection to provide a subjective answer on the quality of the parameters, where the patient can provide a numerical answer on a scale of 0 to 10, with 10 being the best quality). Still further example embodiments of the user interface can provide graphical object representation of parameters (e.g., in a sliding bar interface, colors, pain-scale emojis, or the like).

In the example embodiment of user interface 720a, the user can complete percentage-based answers related to the importance of each parameter to equal a total percentage 719a of 100%. Here, the patient inputs a value of 20% for the pain parameter 712a, 15% for the medication reduction parameter 713a, 25% for the activity parameter 714a, 10% for the mood and depression parameter 715a, 15% for the sleep parameter 716a, and 15% for the mobility parameter 718a. The patient can change the parameter importance percentage values at any time, where a patient-initiated change can cause the patient state monitoring and processing system 130 to reanalyze the parameter inputs and recalculate the patient state.

FIG. 7B similarly depicts the data input user interface 720b as including a number of patient inputs related to the patient-specific goals (e.g., “What are your goals” 701b) and inputs related to providing answers to patient state parameter questions 702b. The user interface 720b can provide the user with methods to enter goals 757b, complete multiple-choice questionnaire 758b, associate external applications 769b, provide additional user input 770b, and/or provide a patient's (or user's) preferred state to the patient state monitoring and processing system 130. The user interface 720b can further provide input capabilities for chronic pain related life data types (e.g., data to be collected) 2050 as described and depicted in connection with FIG. 20.

In either example, the patient answers may be received using other techniques. The user interface may also provide guidance, warnings, validations, or informative information to help gather the input from the user, as well as generate reminder alerts when the system identifies additional data is needed. The questions may be provided in connection with a time-based questionnaire (e.g., a morning questionnaire, an evening questionnaire) that includes a fixed number of prompts (e.g., a series of questions). A questionnaire may be used to determine a particular patient state at one or more times (e.g., a first state at the time that a first program was in use, a second state at the time that a second program was in use, etc.).

Additional example embodiments of the user interfaces 720a, 720b can include other user input functionality, such as menu selection functionality, voice input functionality, text input functionality, and a submit control functionality. A variety of dynamic user interface features may also be used based on validating or entering user data. In other examples, input data may be provided from non-graphical or non-text inputs, such as voice interactions controlled by a chatbot or by virtual assistants or agents (e.g., Amazon® Alexa, Google® Assistant, Apple® Siri, etc.).

Example embodiments of the user interfaces 720a, 720b can provide the patient the ability to modify patient goals at any time (e.g., a weekly basis, monthly basis, etc.), without the need for clinician intervention or doctor appointments. The system can further help identify the specific goals, identify problem areas for achieving goals, and relate the goals and achievements back to the patient in order to provide the patient with encouragement through a process of measuring patient progress and relating tracked goals back to the patient.

Additional example embodiments of different user interfaces can be specifically provided to each of the clinician, doctors, and caregivers, which can include user-specific data. For example, clinicians may desire to set goals for patients so that patients have something to work towards and visualize the benefit of the therapy. Clinicians may further want to set goals for patients that are measurable and achievable.

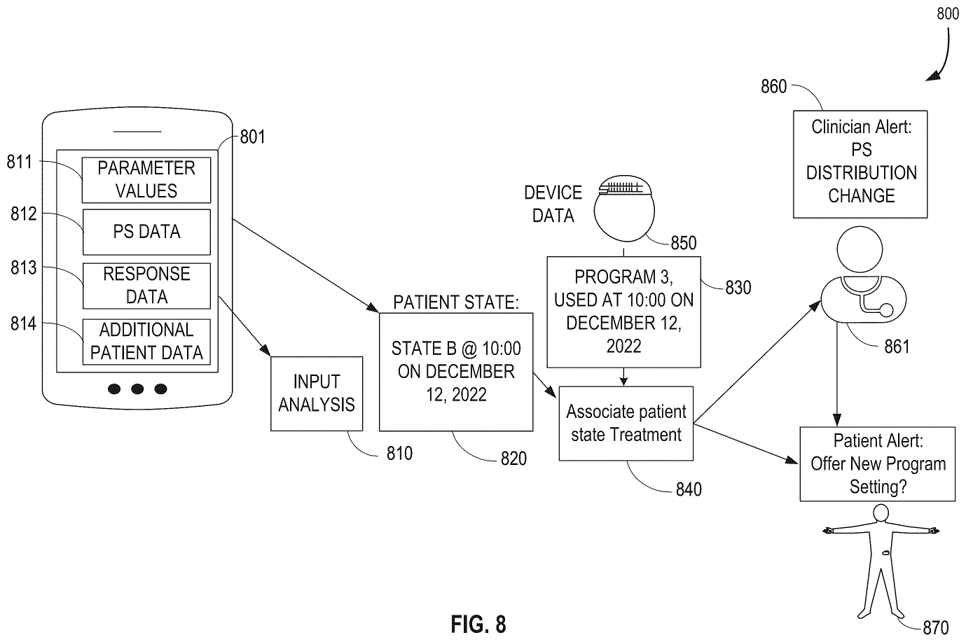

FIG. 8 illustrates, by way of example, data processing operations 800 being performed on patient input entered into a user interface 801 (such as user interfaces 700a, 700b). In the example of FIG. 8, parameter values 811, patient state data 812, response data 813, and/or additional patient data 814 provided from the patient (or from data sources associated with the patient) are evaluated with patient input analysis operations 810, to produce a patient state 820. For instance, the patient state 820 can correspond to a representative health state based on the combined dimensions of the patient's health state (e.g., parameters) captured at or associated with a particular time (e.g., minute, hour, day). The patient state 820 or its derivative can also represent other attributes such as intensity (e.g., the amount of change in patient overall pain health) or be provided as a weighted or composite state.

The questions can be provided in connection with a time-based questionnaire (e.g., a morning questionnaire, an evening questionnaire) that includes a fixed number of prompts (e.g., a series of questions). A questionnaire can be used to determine a particular patient state at one or more times (e.g., a first patient state at the time that a first program was in use, a second patient state at the time that a second program was in use, etc.). In example embodiments, the patient inputs (e.g., answers, values, responses) can be received using other techniques. The user interface 801 can also provide guidance, warnings, validations, or informative information to help gather the input from the user. A variety of dynamic user interface features can also be used based on validating or entering user data. In other examples, input data can be provided from non-graphical or non-text inputs, such as voice interactions controlled by a chatbot or by virtual assistants or agents (e.g., Amazon® Alexa, Google® Assistant, Apple® Siri, etc.). Although the preceding examples refer to the use of individual programs, it will be understood that training data may also be collected from the use of customized programming settings (e.g., changes in amplitude, intensity, waveforms, etc.) that are controlled by a user. Further, training data can be collected after providing recommendations for specific activities (e.g., program changes, programming setting changes, personal activities, etc.) that are delivered via one or more schedules, tasks, and goals.

The patient state 820 provides a data point for a particular patient state at a point in time, which can be associated with a device state at the same point in time. This device state can originate from device data 830 provided from the neurostimulation device 2221. Based on a matching or other correlation of the patient state 820 to the device data 830, a patient state (represented by the valence score) can be associated 840 to a treatment state.

The associated patient and treatment state can be used to trigger an action, such as a clinician alert 860 to a clinician 861 (e.g., device manufacturer representative, doctor, etc.), relating to a neurostimulation program (e.g., indicating that the program used at the point in time is not effective). The clinician alert 860 can provide for faster intervention by a clinician or device rep or other patient intervention via a phone call, text, an in-app message (e.g., educational videos, setting appointments with clinicians, video calls, etc.) and the like. Clinician alert 860 can also be generated to alert the device rep or other caregiver depending on patient system settings.

In the example of FIG. 8, similar operations are performed to determine and initiate relevant actions. Here, parameter values 811, patient state data 812, response data 813, and/or additional patient data 814, originating from the patient or associated with the patient, are evaluated with input analysis operations 810. Although multiple statements are provided in the parameter values 811, patient state data 812, response data 813, and/or additional patient data 814, a single patient state 820 is produced, representing a composite overall pain category for the multiple statements, parameters, or other input. The patient state 820 is compared with a threshold, such as a previous patient state for a specified time period. If the patient state has changed for the negative, or if no change has occurred when a change was desired, an action is taken. Here, the action includes a first action providing a clinician alert 860 that the pain treatment is not effective, and a second action providing a patient alert 870 with an option to implement a new program setting.

Similar aspects of triage, device diagnostics, remediation, or logging can be used based on the detection of such complaints. In some example embodiments, a natural language processing algorithm can be implemented with use of a rule-based sentiment analyzer, such as the VADER (Valence Aware Dictionary for sEntiment Reasoning) model, which uses a list of lexical features (e.g., words) that are positive or negative. This model is sensitive to both polarity (positive/negative) and intensity (strength) of emotion indicated within patient input. It will be understood that other models can be trained or turned with specific consideration of pain treatment and physiological conditions, and analysis or data values from multiple models may also be considered. Further, it will be understood that words can be masked or de-emphasized as part of the analysis. For instance, commonly used words such as “pain” may not be helpful for assessing user sentiment and may require consideration of surrounding words or phrases.

The input analysis 810 can include implementing one or more various types of clustering algorithms, such as hierarchical clustering, DBSCAN, k-means clustering, spectral clustering, or others based on the data being clustered. The input analysis 810 can further be used to perform patient triage to help make it easier for clinicians (including device manufacturer representatives, physicians, etc.) to identify which patients are in need of what triage. The input analysis can also help identify the types and variations of therapies that are effectively meeting patients' treatment needs. It will be understood that the collection text may extend into a variety of settings, being integrated into multiple products/apps, including feedback captured during patients' normal activities, clinician visits, and other events, thus capturing a more realistic view of a patient state.

FIG. 9A illustrates, by way of example and not limitation, a patient state change pathway 900a for identifying an example of a multi-step pathway of state transitions without reaching each state.

For example, each state may have a range of sleep activity or other sleep-related parameters, but in order to get a patient from State E to State A, there is a patient state pathway that is either direct (e.g., going through each state) or indirect (e.g., going through some, but not all states and/or going back to a prior state through an intermediary state). The patient state change pathways can be treatment suggestion options provided to a patient (or other user) to help the patient visualize the specific parameters to change in their life in order to help the patient shift their overall health from a first state to a final state for a specific time period.

The patient state change pathway 900a illustrates an example multi-step pathway of state transitions that results in a patient reaching a final state (e.g., State B 220a) without reaching each individual other consecutive state on the patient's journey to State B 220a. For example, the patient may begin at the current state (e.g., State E 250a) and the patient state monitoring and processing system 130 can determine an ideal pathway for the patient by moving from State E 250a to State D 240a, and next by moving from State D 240a directly to State B 220a without moving to State C.

In order for the patient to accomplish (or attempt) this patient state change pathway, the system suggests one or more parameters to adjust or alter over a period of time in order to reach the next state. For example, in order for the patient to move from State E 250a to State D 240a, the system determines and provides a suggestion 915a that the patient should improve their sleep habits. Next, in order for the patient to move from State D 240a to State B 220a, the system determines and provides a suggestion 913a that the patient should reduce one or more specified medications. In additional example embodiments, the system can provide additional parameter adjustment suggestions in order to reach the next state, including providing sequential parameter changes that occur once the patient has successfully changed the previous parameter.

FIG. 9B illustrates, by way of example and not limitation, a patient state change pathway 900b for identifying a different example of a multi-step pathway of state transitions without reaching each state. The patient state change pathway 900b is similar to the change pathway 900a; however, the example embodiment of FIG. 9B moves from a first state to a last state by transitioning to every other state. For example, the patient state change pathway 900b suggests to the patient starting in State E 250b to improve their mood 915b to reach State C 230b (without first moving to State D). Next, the pathway 900b suggests to the patient to reduce one or more of their current activities 914b to reach State A 210b.

According to some example embodiments, change pathways (or other treatment plan suggestions provided by the system) can utilize patient states to show that the patient has reached a specific state, dwelled in a state for a period of time, reduced the amount of movement between states, and/or succeeded in changing one or more parameters that affect the patient's overall health and/or patient state. Such objective measurements of patient behaviors can help identify parameters and patient states that are most desirable, advantageous, and/or significant in improving the patient's overall life (not just changing an amount of physical pain).

In additional example embodiments, the patient can rate the states for different preferences. For example, patient X may have an increased mood and level of life satisfaction when in State D over State A, so despite State D being a lower-level state, the patient may desire being in that lower state because of their parameter effects. In another example, patient Y may prefer not to go above State B at any time because changing any additional parameters in patient Y's life to reach State A is unwanted. In yet another example, patient Z may prefer to stay in State C at all times because the parameters that define State C enable patient Z to live a satisfactory life without making more changes.

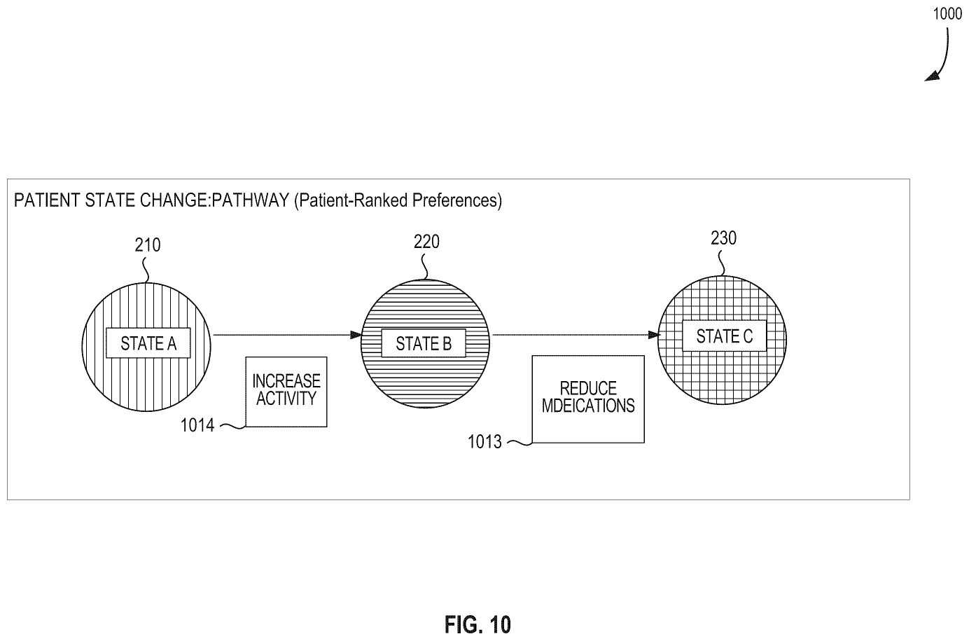

FIG. 10 illustrates, by way of example and not limitation, a patient state change pathway 1000 for identifying a different example of a multi-step pathway of state transitions based on patient-ranked preference on preferred pain state.

In the example change pathway 1000, a patient may have ranked their most preferred (or ideal) patient state to be State C 230. Although the patient is currently in State A 210, the patient may ultimately (or at least for some time period) be interested in joining a gym to increase their activity and reducing one or more medications because the medication side effects are lowering their overall quality of life. Based on the patient's lifestyle desires, the patient (alone, or in conjunction with their care team and/or the closed-loop system described herein) has a preference to be in State C 230. As such, the system suggests that the user first increase their activity 1014 in order to move from current State A 210 to State B 220. Once the patient is in State B 220 for a desired, suggested, and/or appropriate calculated dwell time, the system suggests reducing medications 1013 in order for the patient to move from State B 220 to State C 230.

Changing the parameters (as described and depicted in connection with FIG. 2 and throughout the disclosure) in order to move the patient between different states (e.g., States A-E) may cause obstacles for the patient, such as confounding events, defeating changes, etc. The confounding events, such as reducing medication and/or increasing activity, may cause periods of time where the patient state declines before later improving. Confounding events or variables, also known as confounders, may include variables that can cause or prevent the outcome of interest (e.g., the patient state improving to the global optimal order and/or the patient optimum), and are associated with the parameters and patient states being observed. Such confounders can cause bias in the estimate of the relationship between the parameters (change in parameters) and the outcome (being the global optimal order). In other words, confounding variables can include outside influence that changes the relationship between an independent variable (e.g., cause) and a dependent variable (e.g., effect). Example embodiments of the present disclosure provide control for such confounding variables so as the illusion of a cause-and-effect relationship (where one may not actually exist) is accounted for, as well as accounting for the possibility of a hidden relationship that is actually present.

In additional example embodiments, the system can enable patients to rank the progression of states to their personal preference. For example, a patient may expressly list that their preferred order of states includes State B, State C, State A, State D, then State E. In other words, the system can accommodate for user (e.g., patient preferences or clinician suggestions) preferences over a predetermined order of “best” to “worst” states. Further example embodiments of the patient state change pathway can include identifying a multi-step pathway of state transitions on patient-ranked preference on preferred parameters, objectives, and/or criteria that are used as input into the patient state monitoring and processing system 130.

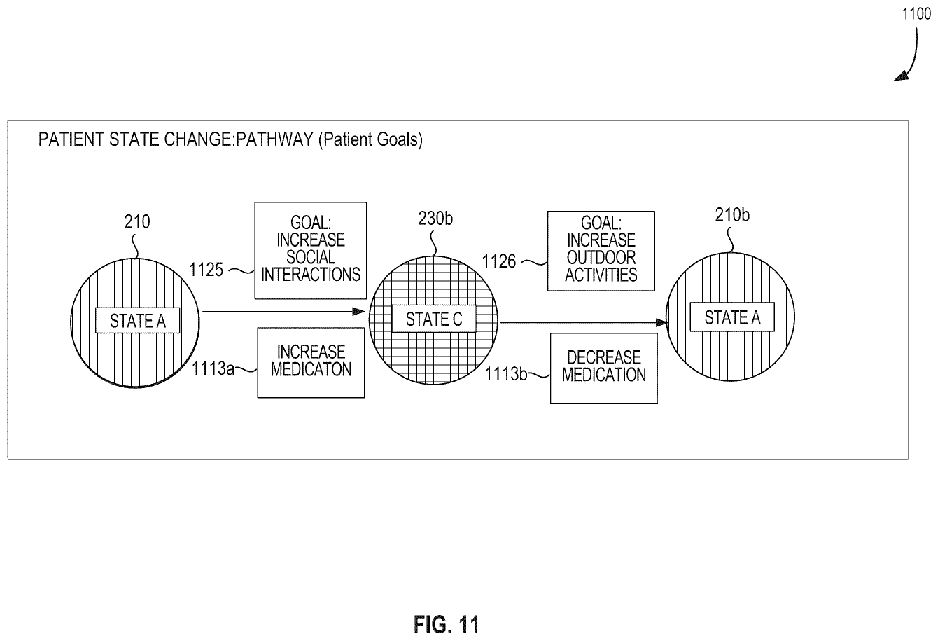

FIG. 11 illustrates a patient state change pathway 1100 for identifying a different example of a multi-step pathway of state transitions based on patient goals, in accordance with some example embodiments. In order for patient states to be defined for each patient, the patient state monitoring and processing system 130 can include receiving data across the domains of importance in order to assign the patient to a cluster. While any or all of the patient-related data may be received, the system 130 can further receive parameters or goals related to parameters in specified orders (e.g., order of importance to be most satisfied overall).

For example, if State A 210 is the ultimate goal for the patient, the pathway can be generated based on any combination of states considering one or more goals set by and/or for the patient. The system 130 can use the patient states to track the patient's goals in order to provide treatment plans and return data to the closed-loop system. For example, the patient's goals can be directed toward patient states achieved, patient state dwell times, distributions, and/or percentages, patient state stability, reduction in patient state transitions, specific parameters used as input to generate the patient state cluster, and/or other goals related to the patient that would improve the patient's overall quality of life.

In the patient state change pathway 1100, the patient's preferences on patient state may be less important than the patient's preferences placed on important activities. While the patient is currently in State A 210 (implying the patient is in the optimal state in some embodiments), the patient may not be satisfied with their life. For example, while the patient is in the best state, the patient cannot visit with fiends or leave their house. Thus, the patient's specific goals are more important than the patient state perceived to be best for the patient.

Based on receiving data related to the patient's goals (e.g., goal 1125 to increase social interaction), the system 130 can suggest that the patient temporarily increase use of one or more medications 1113a in order to achieve or move toward achieving this goal. By increasing medication and also increasing social interactions, the patient's patient state temporarily moves down to State C 230. However, after a period of time in State C, the patient provides data related to further goals (e.g., goal 1126 to increase outdoor activity). The system can provide additional suggestions (e.g., decrease medication 1113b) to the patient in response to the additional goal 1126. As the patient's goals change, the system can adapt to patient-related goals and patient state optimization by making minor changes to different parameters.

Additional example embodiments can include the patient state monitoring and processing system 130 translating patient goals into patient state-based goals. For example, the system can receive input or data that the patient wants to reduce pain sensation and perform more activities. These patient goals can be translated by the system 130, or a component thereof, to equating to a greater than 50% dwell time in State A.

In further example embodiments, the patient may desire to achieve reaching State A 210 with a sustained greater than 75% dwell time in State A. Here, the system 130 can map the patient goal to a patient state dwell time recommendation instead of a specific single state among the patient states; for example, the system can recommend parameters that would cause the patient to be in State A 70% of the time period and State B 30% of the time period. It will be understood by one of ordinary skill in the art that the example embodiments of FIGS. 9A, 9B, 10, and/or 11 can include transitioning between more than three states over the same or different time periods, as well as using the different user and/or system preferences in combination.

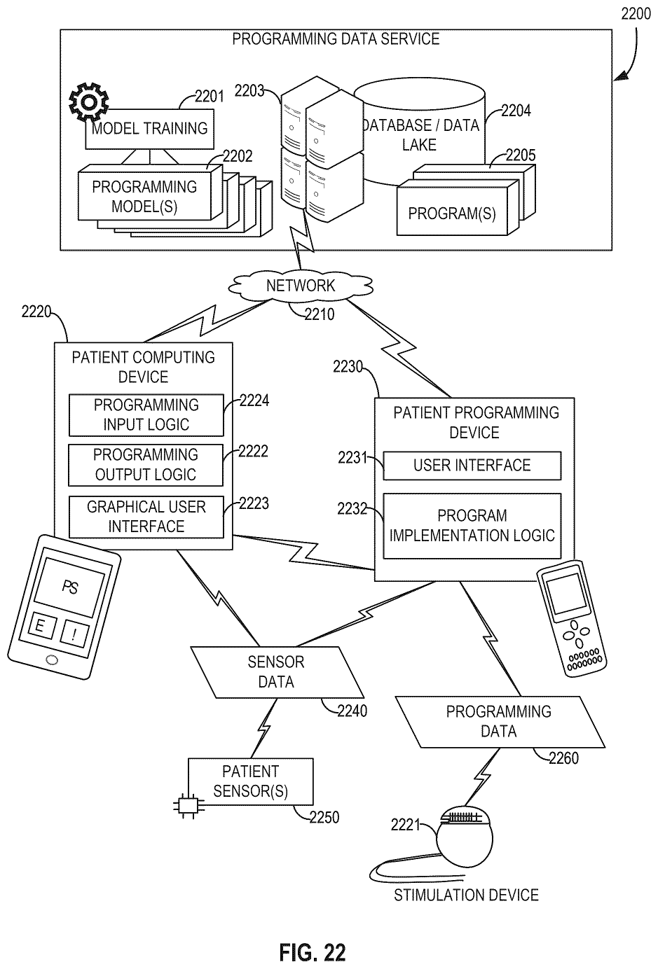

The example embodiments of the patient state change pathways of FIGS. 9A, 9B, 10, and 11 can be implemented as part of patient therapy as one or more recommendations (e.g., ingredients for stimulation parameters) in the programming data service 2200 described and depicted in connection with FIG. 22. For example, the change pathway suggestions can be used for treatment planning decisions, for triage and reprogramming planning and descriptions, for identifying problem areas for achieving goals, and/or additional neurostimulation treatment recommendation options.

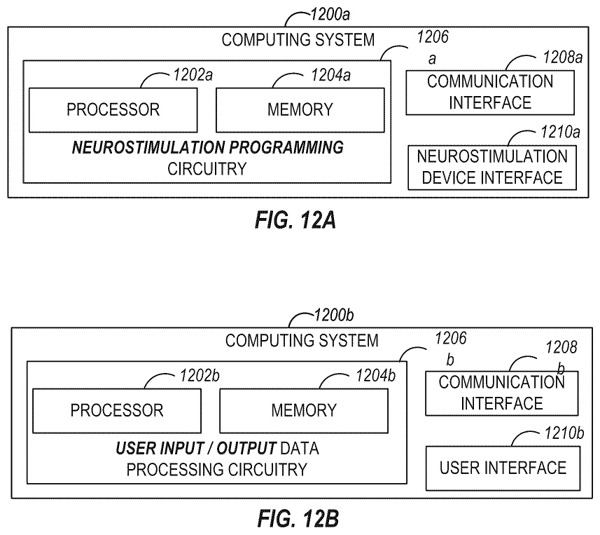

FIG. 12A illustrates, by way of example, a block diagram of an embodiment of a system 1200a (e.g., a computing system) implementing neurostimulation programming circuitry 1206a to cause programming of an implantable electrical neurostimulation device, for accomplishing the therapy objectives in a human subject based on a trained closed-loop programming model as discussed herein.

The system 1200a may be operated by a clinician, a patient, a caregiver, a medical facility, a research institution, a medical device manufacturer or distributor, and embodied in a number of different computing platforms. The system 1200a may be a remote control device, patient programmer device, program modeling system, or other external device, including a regulated device used to directly implement programming commands and modification with a neurostimulation device. In some examples, the system 1200a may be a networked device connected via a network (or combination of networks) to a computing system operating a user interface computing system using a communication interface 1208a. The network may include local, short-range, or long-range networks, such as Bluetooth, cellular, IEEE 802.11 (Wi-Fi), or other wired or wireless networks.

The system 1200a includes a processor 1202a and a memory 1204a, which can be optionally included as part of neurostimulation programming circuitry 1206a. The processor 1202a may be any single processor or group of processors that act cooperatively. The memory 1204a may be any type of memory, including volatile or non-volatile memory. The memory 1204a may include instructions, which when executed by the processor 1202a, cause the processor 1202a to implement the features of the neurostimulation programming circuitry 1206a. Thus, the electronic operations in the system 1200a may be performed by the processor 1202a or the circuitry 1206a.

The processor 1202a or circuitry 1206a may directly or indirectly implement neurostimulation operations including the use of neurostimulation device programming based on a trained programming model. The processor 1202a or circuitry 1206a may further provide data and commands to assist the processing and implementation of the programming using communication interface 1208a or a neurostimulation device interface 1210a. It will be understood that the processor 1202a or circuitry 1206a may also implement other aspects of the device data processing or device programming functionality described above.

FIG. 12B illustrates, by way of example, a block diagram of an embodiment of a system 1200b (e.g., a computing system) for performing analysis of patient feedback data (e.g., patient input data collected as training data in a user interface 1210b) in connection with the data processing operations discussed above.

The system 1200b may be integrated with or coupled to a computing device, a remote control device, patient programmer device, clinician programmer device, program modeling system, or other external device, deployed with neurostimulation treatment. In some examples, the system 1200b may be a networked device (server) connected via a network (or combination of networks) which communicates to one or more devices (clients) using a communication interface 1208b (e.g., communication hardware which implements software network interfaces and services). The network may include local, short-range, or long-range networks, such as Bluetooth, cellular, IEEE 802.11 (Wi-Fi), or other wired or wireless networks.

The system 1200b includes a processor 1202b and a memory 1204b, which can be optionally included as part of user input/output data processing circuitry 1206b. The processor 1202b may be any single processor or group of processors that act cooperatively. The memory 1204b may be any type of memory, including volatile or non-volatile memory. The memory 1204b may include instructions, which when executed by the processor 1202b, cause the processor 1202b to implement data processing, or to enable other features of the user input/output data processing circuitry 1206b. Thus, electronic operations in the system 1200b may be performed by the processor 1202b or the circuitry 1206b.

For example, the processor 1202b or circuitry 1206b may implement any of the features of the method 1600-1900 to obtain and process patient feedback data, to generate user interface displays, and to provide training data for training of a programming selection model. It will be understood that the processor 1202b or circuitry 1206b may also implement aspects of the logic and processing described above, for use in various forms of closed-loop and partially-closed-loop device programming or related device actions.

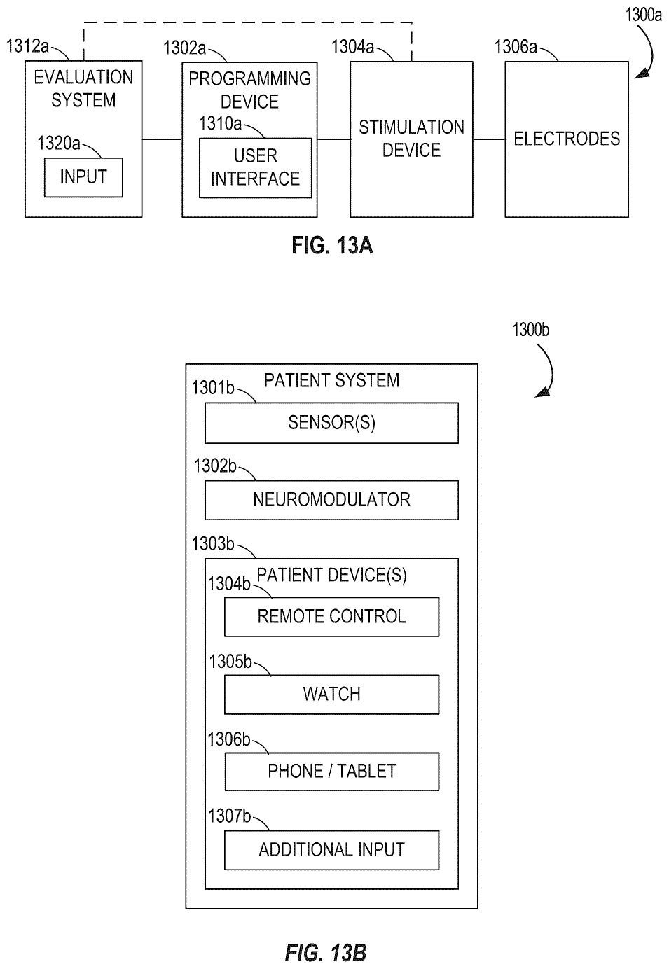

FIG. 13A illustrates an embodiment of a neurostimulation system 1300a.

System 1300a includes electrodes 1306a, a stimulation device 1304a, and a programming device 1302a. Electrodes 1306a are configured to be placed on or near one or more neural targets in a patient. Stimulation device 1304a is configured to be electrically connected to electrodes 1306a and deliver neurostimulation energy, such as in the form of electrical pulses, to the one or more neural targets though electrodes 1306a. The delivery of the neurostimulation is controlled by using a plurality of stimulation parameters, such as stimulation parameters specifying a pattern of the electrical pulses and a selection of electrodes through which each of the electrical pulses is delivered.

In various embodiments, at least some parameters of the plurality of stimulation parameters are selected or programmable by a clinical user, such as a physician or other caregiver who treats the patient using system 1300a; however, some of the parameters may also be provided in connection with closed-loop programming logic and adjustment. Programming device 1302a provides the user with accessibility to implement, change, or modify the programmable parameters. In various embodiments, programming device 1302a is configured to be communicatively coupled to stimulation device 1304a via a wired or wireless link.

In various embodiments, programming device 1302a includes a user interface 1310a (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The present approaches further provide examples of an evaluation system 1312a, such as a data analysis system, which is used to adapt, modify, start, stop, monitor, or identify a neurostimulation treatment with stimulation device 1304a. This evaluation system 1312a initiates an action related to the neurostimulation treatment based on text analysis performed on input text 1320a. This input text 1320a can be directly collected from the patient and analyzed by the evaluation system 1312a, to then cause a programming effect in the programming device 1302a, and the stimulation device 1304a, and the neurostimulation treatment provided by the electrodes 1306a.

A user (e.g., the patient, clinician, device representative, etc.) can provide parameter inputs to the evaluation system 1312a, which are used to select, load, modify, implement, measure, analyze, monitor, and/or evaluate one or more parameters of a defined program for neurostimulation treatment that is implemented by the stimulation device 1304a, or the operation of the stimulation device 1304a. This evaluation can be based on a combination of natural language processing, sentiment analysis, rules, and other operational or treatment objectives that are identified. Various logic or algorithms can then determine an appropriate action to take based on the state of the patient, including but not limited to: a program or parameter change or recommendation to produce an improvement for a treatment objective (such as to address pain, increase mobility, reduce sleep disruption, and the like); diagnostic or remedial actions on the stimulation device 1304a; data logging or alerts to the patient or a clinician associated with the patient; and the like.

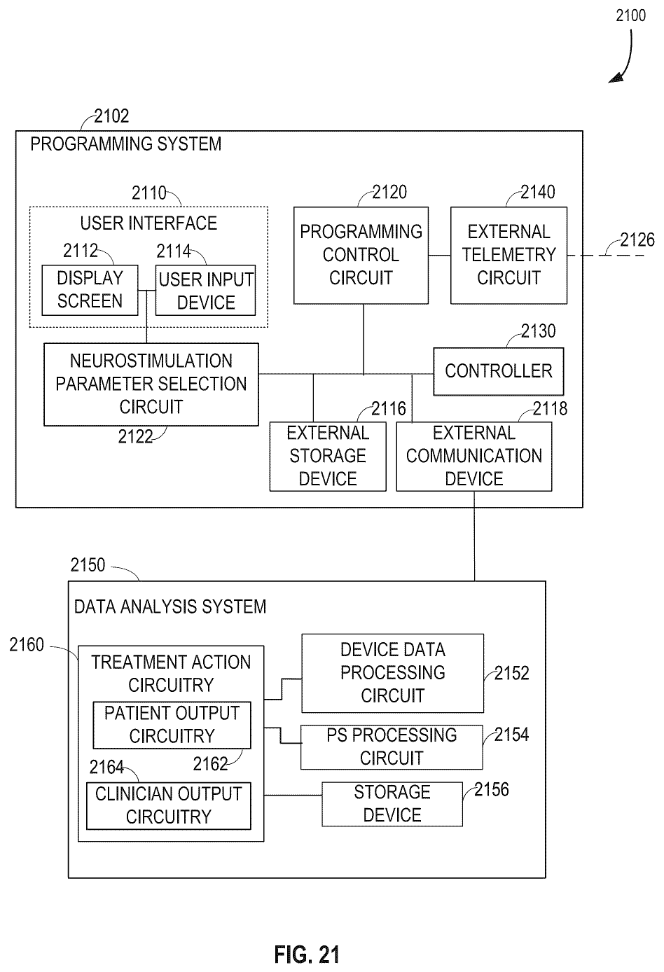

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 21, a controller, e.g., controller 2130 of FIG. 21, can implement program(s) and parameter setting(s) to affect a specific neurostimulation waveform, pattern, or energy output, using a program, or setting in storage (e.g., external storage device 2116 of FIG. 21), or using settings communicated via an external communication device 2118 of FIG. 21 corresponding to the selected program. The implementation of such program(s) or setting(s) may further define a therapy strength and treatment type corresponding to a specific pulse group, or a specific group of pulse groups, based on the specific programs or settings. The evaluation system 1312a and the evaluation of the input text 1320a provides a mechanism to determine the effectiveness of such programs or settings, and to identify issues and provide remediation for ineffective programs or settings, offer suggestions or recommendations for new or updated programs and/or settings, or even to automatically change programs or settings.

Portions of the evaluation system 1312a, the stimulation device 1304a (e.g., implantable medical device), or the programming device 1302a can be implemented using hardware, software, or any combination of hardware and software. Portions of the stimulation device 1304a or the programming device 1302a can be implemented using an application-specific circuit that can be constructed or configured to perform one or more particular functions or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 1300a could also include a subcutaneous medical device (e.g., subcutaneous implantable cardioverter-defibrillator (S-ICD), subcutaneous diagnostic device, wearable medical devices (e.g., patch-based sensing device), or other external medical devices.

FIG. 13B illustrates a patient system 1300b, and examples of devices that may make up components of the patient system, in accordance with some example embodiments. The patient system 1300b may include component(s) that act on the patient such as deliver a therapy to the patient, component(s) used to sense a condition, status or environment of the patient, and component(s) enabling the patient to interface with the patient system.

For example, the illustrated patient system 1300b may include sensor(s) 1301b to sense patient parameter(s). Examples of sensors may include, but not limited to, neural activity, muscle movement, patient activity, patient posture, patient location, breathing, heart rate, blood pressure, temperature, analyte sensors and the like. The illustrated patient system 1300b may include a therapy-delivering device such a neuromodulator 1302b configured to deliver a neuromodulation therapy such as a DBS, SCS, PNS, FES, transcutaneous electrical nerve stimulation (TENS), or other therapy. Those of ordinary skill in the art would understand, upon reading and comprehending the present disclosure, how to apply the present subject matter to other therapies, such as but not limited to cardiac rhythm therapies or drug pump therapies.

The illustrated patient system 1300b may also include device(s) 1303b used by the patient to interface with the patient system. These device(s) may include sensor(s) (e.g., accelerometer, temperature sensor, heart rate sensor, etc.) and/or can be used to receive patient feedback such as responses to questions or free text responses. These device(s) may include interfaces used to interact with the therapy delivery. Examples of patient device(s) 1303b include, but are not limited to, a patient remote control 1304b, a wearable device(s) such as a watch 1305b, or a phone or tablet 1306b, or another personal device or additional input 1307b.

FIG. 14 is a block diagram 1400 illustrating three patient state cluster solutions 1401, 1402, 1403 using a variety of adaptive techniques to determine patient states, in accordance with some example embodiments.

According to some example embodiments of the present disclosure, for a particular version of patient states based on a particular cluster solution, the patient state monitoring and processing system 130 can include receiving a specific set of data streams (e.g., parameters) for computation of each cluster solution (e.g., heat map). However, sometimes patients may miss or choose not to provide a data stream, which can prevent a state from being properly computed without additional imputation. In some example embodiments, the system 130 can include data imputation, which refers to a process of replacing missing data with substituted values, such as can be used to prevent missing data from introducing bias into the model(s) or machine learning.

In the example embodiment of FIG. 14, the patient state monitoring and processing system 130 can identify a need for patient state adaptation when a data stream (e.g., parameter input) is missing and/or withheld. The system implements patient state adaptation to adjust to available data to compute one or more cluster assignments for a patient state using missing data imputation, adaptive device technology data, degenerating to different state definitions (e.g., less granularity, three states, five states, seven states, etc.), or a combination thereof.

At patient state cluster solution 1401, the system 130 can identify the need to adapt to different patient state solutions to reduce the patient burden by degenerating after a period of missing data and shifting to a patient state solution with lower granularity. For example, if the system detects missing watch data, it can automatically transition 1404b from cluster solution 1401 to cluster solution 1402. The system can also bypass the cluster solution degeneration by transitioning 1404a directly to cluster solution 1403.

At patient state cluster solution 1402, the system 130 adapts after a period of missing data to a three-state patient state cluster solution. For example, when an adaptive device, such as a watch or other monitor is disconnected or failing to provide watch data, one or more states in the five-state patient state cluster solution may not be calculable. As such, the patient state cluster solution 1402 only includes three states, State A′ 210b, State C′ 230b, and State E′ 250b. When fewer states are defined according to a cluster solution, the system 130 can map between multiple patient state formulations for data interpretability. For example, States A and B map to State A′ 210b, State C maps to State C′ 230b, and States D and E map to State E′ 250b. When the system 130 determines additional data may be useful in further defining a patient state cluster solution, it can automatically transition 1404c from cluster solution 1402 to cluster solution 1403.

At patient state cluster solution 1403, the system 130 adapts after a period of missing data (e.g., no associated watch data received) to asking one or more new subjective questions (e.g., via an alert or user interface) to take place of the watch data. Here, the system 130 can revert to a five-state solution, where one or more of the states are generated using additional or new questions on patient activity to compensate for missing or withheld data.

In additional example embodiments, the system 130 can automatically adapt to new states (e.g., States F, G, etc.) if a new state is discovered by the programming data service 2200 (or component thereof, such as model training 2201). The programming data service 2200 can be a closed-loop service, an open-loop service, or a combination thereof. The system 130 can revert to one or more higher state count solutions when a data threshold is exceeded. The patient state monitoring and processing system 130 can further include adaptive measures being automatically implemented where thresholds are adaptive to patient's condition(s) (e.g., during weather changes, menstrual cycles, etc.).

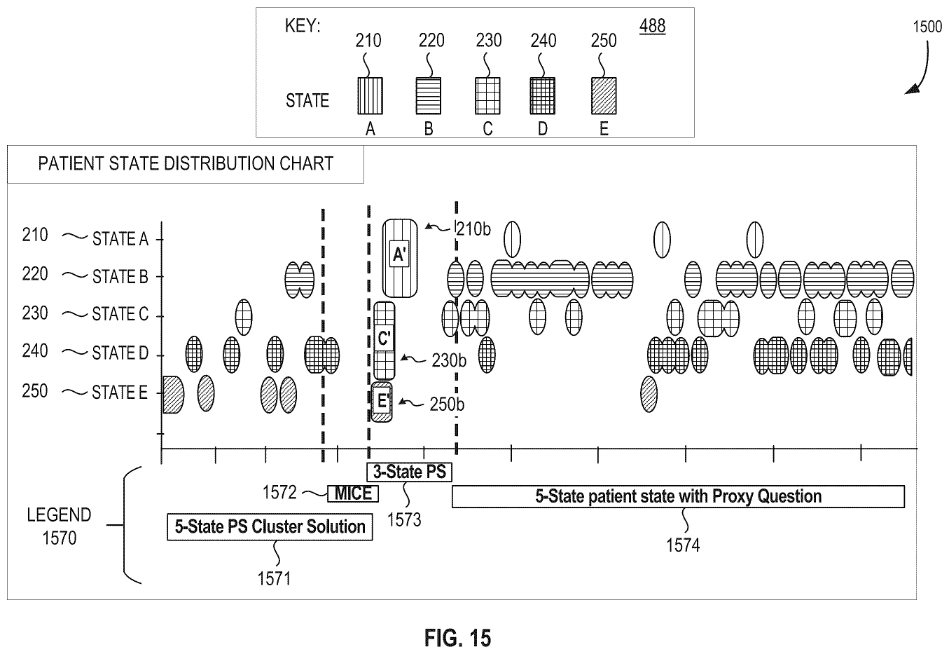

FIG. 15 illustrates a patient state distribution chart 1500 displaying patient states over time through transitions of different solutions using adaptation techniques, in accordance with one example embodiment. The patient state distribution chart 1500 as depicted shares similar features and components as the patient state distribution charts 300 as illustrated and described in connection with FIG. 3. For brevity, only specific elements are detailed and described with reference to FIG. 15.

The patient state monitoring and processing system 130 can process patient parameter input and generate the patient state distribution chart 1500 for providing patient state data plotted over time. The patient state distribution chart 1500 can be one plot used to display patient states over time through transitions of different cluster solutions given that patient state solutions can retain ordinal or rank correlation. For example, the legend 1570 includes four different patient state cluster solutions using a variety of adaptive techniques (as described in detail in connection with FIG. 14, above).

During a first time period, a five-state cluster solution 1571 is used, such as cluster solution 1401. A second time period is shown, which is a transition time period between the five-state cluster solution 1571, and a three-state cluster solution 1573. During the second, transition, time period, the system can generate the patient state clusters using multiple imputation by chained equations (MICE) 1572 to substitute for missing or withheld data. During the third time period, the three-state cluster solution 1573 is used, similar to cluster solution 1402 generated without adaptation. As in FIG. 14, the three-state cluster solution 1573 includes three states (instead of five states), State A′ 210b, State C′ 230b, and State E′ 250b. During a fourth, and last, time period, the system returns to using a five-state cluster solution 1574 including one or more new questions as a proxy for missing data in the effective mobility parameter.

Example embodiments of the present disclosure incorporating patient state adaptation techniques provide use of states in the patient state monitoring and processing system 130 at varying levels of patient compliance with answering parameter input questions (e.g., questionnaires) and/or with wearing system associated data devices (e.g., watch, monitor). Adaptation techniques provide for improvements in patient monitoring that reduce gaps in patient state computation.

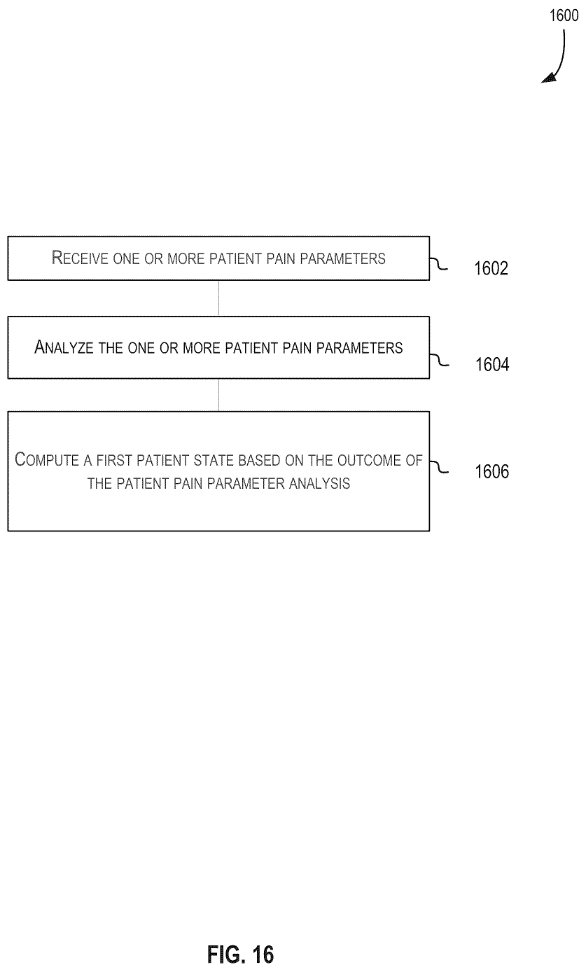

FIG. 16 is a flowchart showing one example of a process flow 1600 (e.g., processing method) for computing patient states for remote triaging of chronic pain patients. The embodiment of the process flow 1600 can be implemented by a system or device for use to analyze, monitor, and correlate health-related data for neurostimulation programming (e.g., in connection with monitoring patient state changes for triaging pain patients using a closed-loop neurostimulation treatment). For example, the process flow 1600 can be embodied by electronic operations performed by one or more computing systems or devices (including those at a network-accessible remote service) that are specially programmed to implement data monitoring, data analysis, and/or neurostimulation data processing operations. In specific examples, the operations of the method 1600 can be implemented through the systems and data flows depicted throughout the disclosure, at a single entity or at multiple locations. For example, the process flow 1600 can be performed by the patient state monitoring and processing system 130.

At operation 1602, the monitoring and processing system receives one or more patient pain parameters. At operation 1604, the monitoring and processing system analyzes the one or more patient pain parameters. At operation 1606, the monitoring and processing system computes a first patient state based on the outcome of the patient pain parameter analysis.

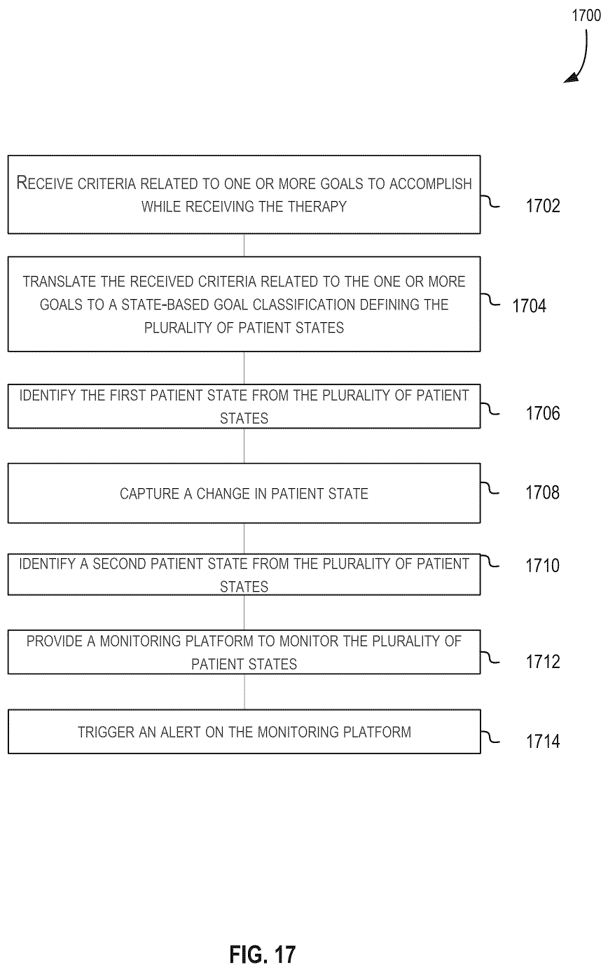

FIG. 17 is a flowchart showing one example of a process flow 1700 for providing a patient state monitoring platform for use with a state-based goal classification system. The process flow 1700 is described as being performed by a closed loop programming data service 2200, although in some examples some or all of the process flow 1700 can be executed by the patient computing device 2220 via a network 2210.

At operation 1702, the data service receives criteria related to one or more goals to accomplish while receiving the therapy. At operation 1704, the data service translates the received criteria related to the one or more goals to a state-based goal classification defining the plurality of patient states. At operation 1706, the data service identifies the first patient state from the plurality of patient states.

At operation 1708, the data service captures a change in patient state. At operation 1710, the data service identifies a second patient state from the plurality of patient states. At operation 1712, the data service provides a monitoring platform to monitor the plurality of patient states. At operation 1714, the data service triggers an alert on the monitoring platform.

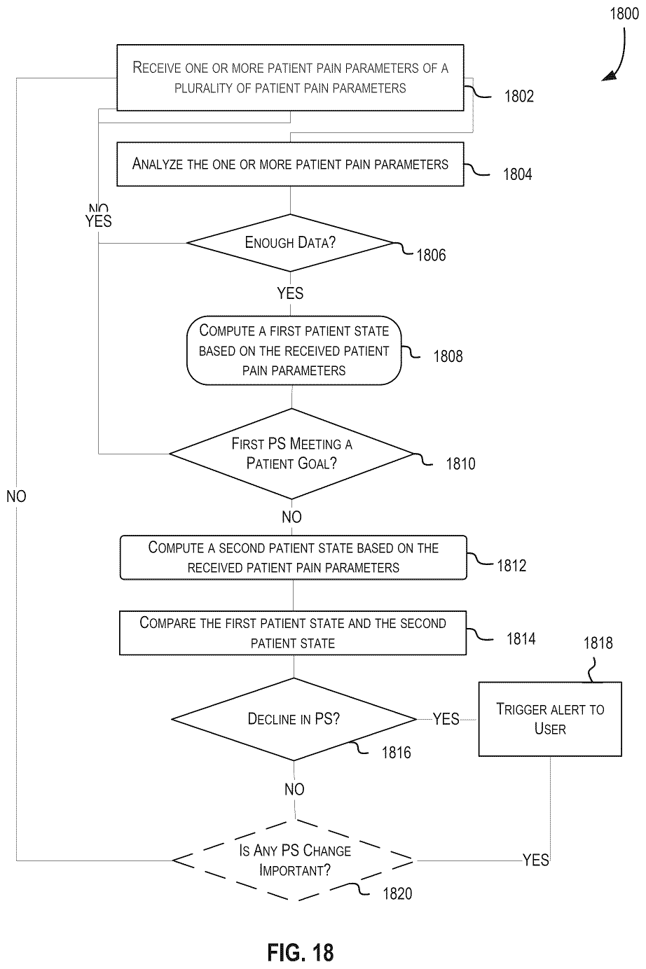

FIG. 18 is a flowchart showing one example of a process flow 1800 for triggering alerts relating to changes in patient states, in accordance with example embodiments. The process flow 1800 can be embodied by electronic operations performed by one or more computing systems, such as the computing system 1200a or 1200b or devices (including those at a network-accessible remote service) that are specially programmed to implement data monitoring, data analysis, and/or neurostimulation data processing operations.

At operation 1802, the computing system receives one or more patient parameters from a plurality of patient parameters. At operation 1804, the computing system analyzes the one or more patient parameters. At operation 1806, the computing system determines if enough data associated with patient pain parameters has been received from the one or more data input devices. If the computing system determines no, not enough data has been received, the process 1800 returns to operation 1802 for continued monitoring. If yes, then, at operation 1808, the computing system computes a first patient state based on the received patient pain parameters.

Returning to the process 1800, at operation 1810, the computing system determines if the first patient state meets a goal associated with the patient. If yes, the computing system maintains the patient state and the process 1800 returns to operation 1802 for continued monitoring. If, at operation 1810, the computing system determines no, the first patient state does not meet a goal associated with the patient, the process 1800 continues. At operation 1812, the computing system computes a second patient state based on the received patient pain parameters. At operation 1814, the computing system compares the first patient state and the second patient state.

At operation 1816, the computing system determines if there has been a decline in the patient state. If yes, the computing system determines that there has been a deterioration or decline in patient state, operation 1818 triggers an alert to the user. If no, the computing system determines that there has not been a decline in patient state, the method 1800 continues to optional operation 1820 to determine if any patient state change is important for the specific patient. If yes, the computing system continues to operation 1818 and triggers an alert despite no decline in patient state. If no, the process 1800 returns to operation 1802 for continued monitoring.

FIG. 19 is a flowchart showing one example of a process flow 1900 for using adaptive input parameters from external data sources to compensate for missing data in patient state monitoring, in accordance with example embodiments. The process flow 1900 can be embodied by electronic operations performed by one or more computing systems, such as the neurostimulation system 1300a or patient system 1300b (including those at a network-accessible remote service) that are specially programmed to implement data monitoring, data analysis, and/or neurostimulation data processing operations.

At operation 1902, the computing system receives a plurality of patient pain parameters from a pain patient. At operation 1904, the computing system correlates the plurality of patient pain parameters with one or more patient states. At operation 1906, the computing system identifies missing data from the plurality of patient pain parameters. For example, missing data may include missing values for one or more parameters (e.g., medication parameter 213, activities of daily living parameter 214, sleep parameter 216, etc.) as described and depicted in connection with FIG. 2. When values (e.g., patient input) are missing, adaptive techniques or technologies (e.g., wearable devices, internal and/or external sensors, environmental data, therapeutic data provided by a clinician, doctor, caregiver, friend or the like, etc.) can be used to provide a mapping between underlying values and/or underlying parameters at two or more time periods.

At operation 1908, the computing system receives adaptive input parameters from one or more external devices. At operation 1910, the computing system associates the patient pain parameters and the adaptive input parameters with a first patient state. At operation 1912, the computing system triggers an alert based on the first patient states. At operation 1914, the computing system provides an updated treatment plan based on the first patient state.

FIG. 20 illustrates a block diagram 2000, by way of example and not limitation, including a patient system 2002 for monitoring patient states and configured to determine, from the health-related information, that patient issue(s) can be adversely affecting the patient treatment plan. The patient state monitor can collect different chronic pain related life data types 2050, which can include, for example, objective data 2051 and/or subjective data 2052.

Objective data is data that can be observed using human senses and can be obtained from a measurement or direct observation. Objective data can be measured by a sensor and can be provided via user input when the user has access to objectively determined information. Examples of objective data can include physiological parameter data 2053, therapy data 2054, device data 2055, environmental data 2056, and/or additional data (e.g., health related data). By way of example and not limitation, physical parameter data 2053 can include data such as: heart rate, blood pressure, respiration rate, activity, posture, electromyograms (EMGs), neural responses such as evoked compound action potentials (ECAPS), glucose measurements, oxygen levels body temperature, oxygen saturation, and gait. By way of example and not limitation, therapy data 2054 can include: neuromodulation programs, therapy on/off schedule, dosing, neuromodulation parameters such as waveform, frequency, amplitude, pulse width, period, therapy usage and therapy type. By way of example and not limitation, device data 2055 can include: battery information (voltage, charge state, charging history if rechargeable), impedance data, faults, device model, lead models, MRI status, Bluetooth connection logs, connection history with Clinician's Programmer (CP). By way of example and not limitation, environmental data 2056 can include: temperature, air quality, pressure, location, altitude, sunny, cloudy, precipitation, etc. By way of example, additional data 2058 can include: healthcare-related data (e.g., menstrual cycles, surgeries, procedures, acute conditions, other non-pain chronic conditions, etc.), lifestyle-related data (e.g., relationship problems, financial problems, at-home stressors, at-work stressors, etc.), and the like.

Subjective data can include information received from the patient. For example, the patient's quantification of pain is a subjective data. Subjective data can generally involve user-inputted data. Examples of subjective data 2052 include questions with free text answers 2057, multiple choice questions 2058, question tree(s) 2059, and/or additional data 2070. Other data can be stored and/or transferred, including detected event(s) 2060, contextual data 2061 for other collected data and/or event(s), and a clock/time 2062 such as can be used to provide a time stamp associated with the retrieved data. The event(s), context(s), and time can be detected by the system or can be provided via user input.

The collected data can be processed, as generally illustrated at data processing 2063. The data processing 2063 can occur in a medical device or a patient device such as a phone, tablet, or remote control, or can occur in a remote data receiving system. The data processing 2063 can include one or more model(s) 2064. The model(s) 2064 can be used to determine how the patient data is used to determine issues (e.g., “common issues”) for which automatic triage alerts 2069 can be beneficial to the user (e.g., the patient, the clinician, the device representative, the patient caregiver, etc.). The model(s) 2064 can be used to determine how the patient data is used to classify the patient state (e.g., disease progression, updated patient goals, change of state, etc.) or the patient interaction, determine the type and contact of intervention, determine issues (e.g., “common issues”) for which automatic patient assistance can be beneficial, determine content for the patient assistance, determine if automatic triage alerts are beneficial, determine if treatment plan updates are beneficial, and the like. The model(s) 2064 can use the acquired data to deliver therapy. Machine learning 2065 (or other artificial intelligence) can be implemented on the collected data to develop or refine the model(s) 2064. The data processing 2063 can include data imputation such as can be used to prevent missing data from introducing bias into the model(s) or machine learning.

FIG. 21 illustrates a block diagram 2100 of a programming system 2102 used as part of an implantable neurostimulation system, such as the external system 2414, with the programming system 2102 configured to send and receive device data (e.g., commands, parameters, program selections, information), in accordance with example embodiments. FIG. 21 also illustrates an embodiment of a data analysis computing system 2150, communicatively coupled to the programming system 2102, with the data analysis computing system 2150 used to perform data analysis on freeform text and device data in connection with neurostimulation treatment by the implantable neurostimulation system.

The programming system 2102 represents an embodiment of the programming device 1302a, and includes an external telemetry circuit 2140, an external storage device 2116, a programming control circuit 2120, a user interface device 2110, a controller 2130, and an external communication device 2118, to effect programming of a connected neurostimulation device. The operation of the neurostimulation parameter selection circuit 2122 enables selection, modification, and implementation of a particular set of parameters or settings for neurostimulation programming. The particular set of parameters or settings that are selected, modified, or implemented can be based on the parameters 211 such as described and depicted with reference to the in FIGS. 2, 7A, 7B, and 11.

The external telemetry circuit 2140 provides the closed loop programming system 2102 with wireless communication to and from another controllable device such as the implantable stimulator 2221 including transmitting one or a plurality of stimulation parameters (e.g., selected, identified, or modified stimulation parameters of a selected program) to the implantable stimulator 2221, via programming data 2260. In one embodiment, the external telemetry circuit 2140 also transmits power to the implantable stimulator 2221 through inductive coupling.

The external communication device 2118 can provide a mechanism to conduct communications with a programming information source, such as a data service, program modeling system, to receive program information, settings and values, models, functionality controls, or the like, via an external communication link (not shown). In a specific example, the external communication device 2118 communicates with the data analysis computing system 2150 to obtain commands or instructions in connection with parameters or settings that are selected, modified, or implemented based on freeform text analysis from the data analysis computing system 2150. The external communication device 2118 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 2140 and the external communication device 2118 are depicted as separate components within the closed-loop programming system 2102, the functionality of both of these components can be integrated into a single communication chipset, circuitry, or device.

The external storage device 2116 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses and may include one or more other waveforms of the plurality of individually definable waveforms. Examples of such waveforms include pulses, pulse blocks, pulse trains, and train groupings, and programs. The existing waveforms stored in the external storage device 2116 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 2116 may also store a plurality of individually definable fields that can be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program can be correlated to the control of these waveforms and definable fields.

The programming control circuit 2120 represents an embodiment of a control circuit and can translate or generate the specific stimulation parameters or changes which are to be transmitted to the implantable stimulator 2221, based on the results of the neurostimulation parameter selection circuit 2122. The pattern can be defined using one or more waveforms selected from the plurality of individually definable waveforms (e.g., defined by a program) stored in an external storage device 2116. In various embodiments, the programming control circuit 2120 checks values of the plurality of stimulation parameters against safety rules to limit these values within constraints of the safety rules. In one embodiment, the safety rules are heuristic rules.

The user interface device 2110 represents an embodiment of the user interface devices 700a and 700b and allows the user (e.g., a patient or clinician) to provide input relevant to therapy objectives, such as to switch programs or change operational use of the programs. The user interface device 2110 includes a display screen 2112, a user input device 2114, and can implement or couple to the parameter selection circuit 2122, or data provided from the data analysis computing system 2150. The display screen 2112 can include any type of interactive or non-interactive screens, and the user input device 2114 can include any type of user input devices that supports the various functions discussed in this document, such as a touchscreen, keyboard, keypad, touchpad, trackball, joystick, and mouse. The user interface device 2110 may also allow the user to perform other functions where user interface input is suitable (e.g., to select, modify, enable, disable, activate, schedule, or otherwise define a program, sets of programs, provide feedback, or input values, or perform other monitoring and programming tasks). Although not shown, the user interface device 2110 may also generate a visualization of such characteristics of device implementation or programming and receive and implement commands to implement or revert the program and the neurostimulator operational values (including a status of implementation for such operational values). These commands and visualization can be performed in a review and guidance mode, status mode, or in a real-time programming mode.

The controller 2130 can be a microprocessor that communicates with the external telemetry circuit 2140, the external communication device 2118, the external storage device 2116, the programming control circuit 2120, the parameter selection circuit, and the user interface device 2110, via a bidirectional data bus. The controller 2130 can be implemented by other types of logic circuitry (e.g., discrete components or programmable logic arrays) using a state machine type of design. As used in this disclosure, the term “circuitry” should be taken to refer to discrete logic circuitry, firmware, or to the programming of a microprocessor.

The data analysis computing system 2150 is configured to operate treatment action circuitry 2160, which can produce or initiate certain actions on the basis of device data (received and processed by device data processing circuit 2152) and input text (received and processed by patient state processing circuit 2154). The treatment action circuitry 2160 can identify one or more actions related to the neurostimulation treatment and provide outputs to a patient or a clinician using patient output circuitry 2162 or clinician output circuitry 2164, respectively. Such outputs and actions provided by the outputs are based on the evaluation and detection of particular patient state(s), triage of patient state(s), and/or device states from text and associated device data.

The data analysis computing system 2150 also is depicted as including a storage device 2156 to store or persist data related to the device data, text input, patient or clinician output, and related settings, logic, or algorithms. Other hardware features of the data analysis computing system 2150 are not depicted for simplicity but are suggested from functional capabilities and operations in the following figures.

As will be understood, patients who are experiencing chronic pain are often willing to provide detailed information regarding their current medical state within varying input options (e.g., freeform text answers to questions, questionnaires, question trees, associated applications, adaptation device data, etc.). For example, freeform text in the form of a narrative, explanatory statement, or interjection is easy for patients to produce, and can provide many details regarding a patient's actions, physiological, and psychological state, prior historical events, and can reflect both objective and subjective results of neurostimulation treatment. Freeform text, however, can be time-consuming or difficult for physicians and clinicians to interpret, especially when patient feedback can be contradictory (e.g., “I felt good in the morning but was unable to do any activity”) or is incomplete without additional context (e.g., “I was unable to get out of bed”). Capturing patient feedback with the present systems can provide many new data points for treatment outcomes, can triage patients using alert notifications, and provide a basis for determining whether or why a particular neurostimulation treatment (and treatment program, programming value, programming effect) is or is not effective.

FIG. 22 illustrates, by way of example, an embodiment of data interactions among a monitoring and processing system implemented as a programming data service 2200, for operation of a stimulation device 2221 with use of closed loop programming, open loop programming, or a combination thereof. At a high level, a programming data service 2200 uses a trained model (one of programming models 2202) to generate programming settings and parameters (e.g., in one or more programs 2205) that are customized to the patient. The programming data service 2200 includes compute hardware 2203 to control the model training 2201 and other data processing operations, such as to generate or control diagnostic actions, alerts, programming recommendations, or programming actions. The programming settings and parameters may be implemented automatically or manually on the stimulation device 2221 (e.g., using the programming techniques referenced above) or via a patient computing device 2220 or patient programming device 2230.

The programming data service 2200 communicates with one or both of the patient computing and programming devices 2220, 2230, to obtain training data for use by model training logic 2201. The training data may be stored in a database 2204 or another large-scale data store (e.g., data lake) for the patient or a population of patients. The programming data service 2200 may also include data analysis or processing engines (not shown) that parse and determine a state of a particular patient from various inputs and correlated program usage (e.g., to determine what programs and programming settings are beneficial or not beneficial to the patient). In some examples, the state of treatment may be based on correlating the historical use of a neurostimulation program or set of parameters with the current state of a patient (e.g., identifying that a pain condition became worse or better after beginning use of a particular program).

The programming data service 2200 can also analyze a variety of forms of patient input and patient data related to usage of a neurostimulation program or neurostimulation programming parameters. For instance, the programming data service 2200 can receive information from program usage, questionnaire selections, text input originating from a human patient, and the like via the patient computing and programming devices 2220, 2230. In addition to providing recommended programs, the programming data service 2200 can also provide therapy content, triage alerts, and usage recommendations to the patient computing and programming devices 2220, 2230.

A patient can provide training data (e.g., input data) via the patient computing device 2220 or the patient programming device 2230. Additional detail of how input data is collected is discussed with reference to the data processing logic and user interfaces discussed in related patent application. In an example, the patient computing device 2220 is a computing device (e.g., personal computer, tablet, smartphone) or other form of user-interactive device which receives and provides interaction with a patient using a graphical user interface 2223, with use of programming input logic 2224 and programming output logic 2222. For instance, the programming input logic 2224 can receive input from a patient via questionnaires, surveys, messages, or other inputs. The inputs may provide text related to pain or overall health, which can be used to identify a psychological state(s), physical state(s), physiological state(s), somatic state(s), or a combination of states of the patient, neurostimulation treatment results, or related conditions. As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

A patient programming device 2230 is depicted as including a user interface 2231 and program implementation logic 2232. The program implementation logic 2232 specifically can provide the patient with the ability to implement or switch to particular programs generated by programming data service 2200. Other forms of closed-loop programming can also include the receipt of instructions, recommendations, or feedback (including clinician recommendations, behavioral modifications, etc., selected for the patient) that are automatically selected based on detected conditions.

The programming data service 2200 can also utilize sensor data 2240 from one or more patient sensors 2250 (e.g., wearables, sleep trackers, motion tracker, implantable devices, etc.) among one or more internal or external devices. The sensor data 2240 can be used to determine a customized and current patient state or neurostimulation treatment results. In various examples, the stimulation device 2221 also includes sensors that contribute to the sensor data 2240 to be evaluated by the programming data service 2200.

In an example, the patient sensors 2250 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

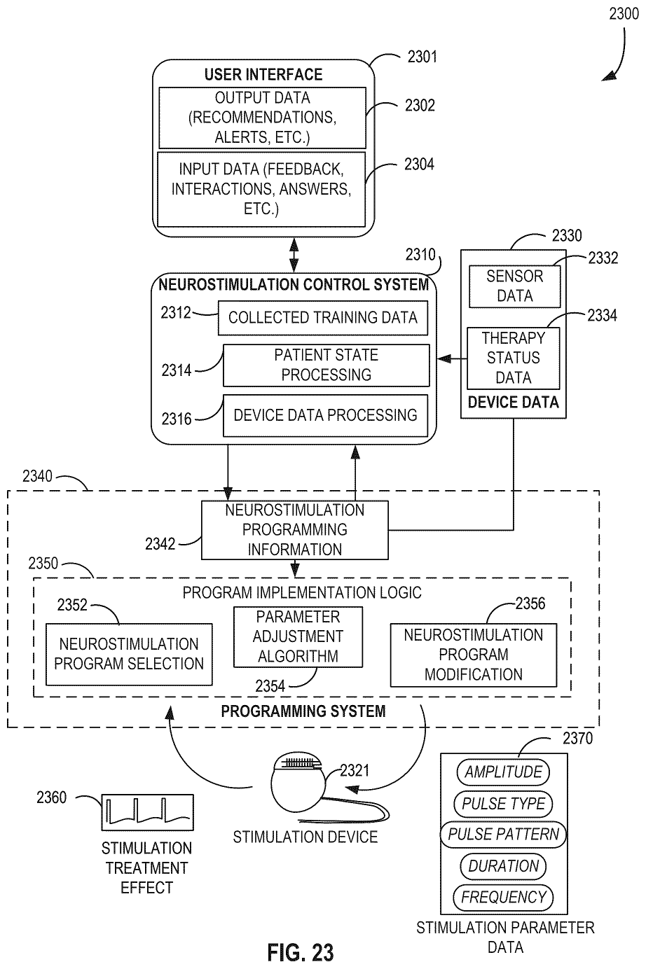

FIG. 23 illustrates, by way of example, an embodiment of a data processing flow 2300 affecting the neurostimulation treatment of a patient, including a neurostimulation control system 2310 based on collected training data 2312, patient state processing 2314, and device data processing 2316 functions. Here, additional details are provided on the data flow between the patient state monitoring and processing system 130 and an example user interface 2301 (e.g., graphical user interface 700a). Other user interfaces and actions are not depicted for simplicity.

In this example, input data 2304 (e.g., parameter input, questionnaire answers, question trees, freeform text, patient feedback, interaction results, etc.) is obtained by the patient state monitoring and processing system 130 from user interface 700a. FIG. 23 also depicts the evaluation of device data 2330, such as sensor data 2332, therapy status data 2334, and other treatment aspects which can be obtained or derived from the neurostimulation device or related neurostimulation programming. Also in this example, output data 2302 (e.g., content) is obtained from the patient state monitoring and processing system 130 at user interface 700a, such as in the form of recommendations, alerts, triage messages, imputation suggestions, treatment updates, patient information, and the like. The patient state monitoring and processing system 130 can separately provide clinician recommendations, clinician alerts 2328, or other related actions that are provided to the clinician, doctor, or device representative separately from the patient.

The remainder of the data processing flow 2300 illustrates how data processing results from the patient state monitoring and processing system 130 can be used to effect programming, such as in a closed loop (or partially-closed-loop) system. A programming system 2340 can use parameters or program information 2342 provided from the patient state monitoring and processing system 130 as an input to program implementation logic 2350. The program implementation logic 2350 can be implemented by a parameter adjustment algorithm 2354, which affects a neurostimulation program selection 2352 or a neurostimulation program modification 2356. For instance, some parameter changes can be implemented by a simple modification to a program operation; other parameter changes may require a new program to be deployed. The results of the parameter or program changes or selection results in definition or adjustment to various stimulation parameters at the neurostimulation device 2321, causing a different or new stimulation treatment effect 2360.

By way of example, stimulation parameter data 2370 includes operational parameters of the neurostimulation device, which are generated, identified, and/or evaluated by the present systems and techniques can include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output and apply any number of control techniques to modify the electrical simulation applied to anatomical sites or systems related to pain or analgesic effect.

In various embodiments, a neurostimulator program can be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

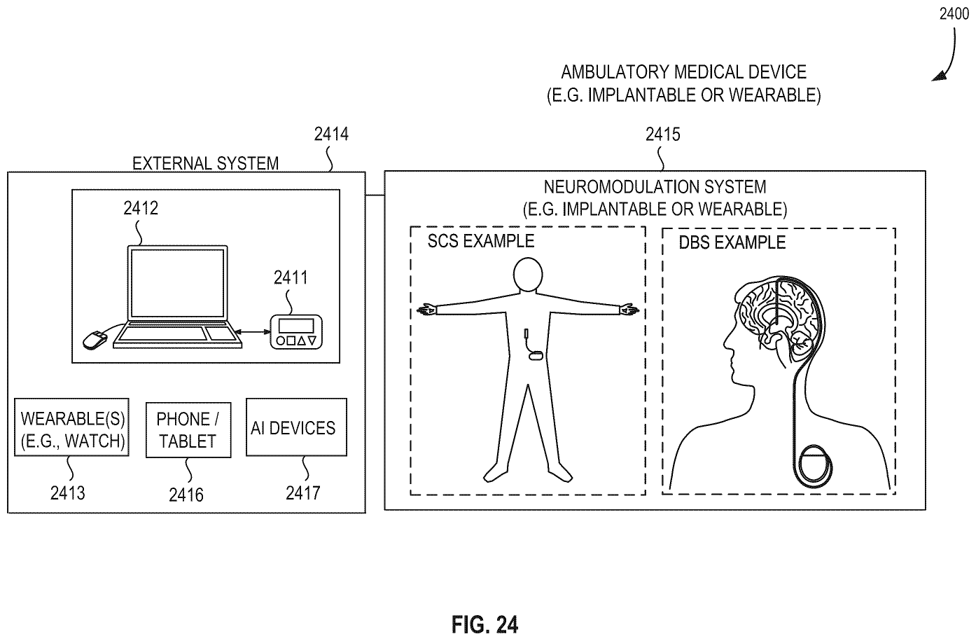

FIG. 24 illustrates, by way of example and not limitation, a block diagram 2400 of the neuromodulation system 2415 of FIG. 13A implemented in a spinal cord stimulation (SCS) system or a deep brain stimulation (DBS) system, example embodiments of the present disclosure can include additional forms of stimulation in a patient. The illustrated neuromodulation system 2415 includes an external system 2414 that can include at least one programming device. The illustrated external system 2414 can include a programmer 2411 configured for use by a clinician, patient, device representative, or other caregiver to communicate with and program the neuromodulator, and a remote control (not shown) configured for use by the patient to communicate with and program the neuromodulator.

For example, the remote-control device can allow the patient to turn a therapy on and off and/or can allow the patient to adjust patient-programmable parameter(s) of the plurality of modulation parameters. The external system 2414 can further be operatively coupled with one or more patient wearable devices 2413 (e.g., watch, ring, brain-sensing monitor, necklace, heartrate monitor, Holter monitor, etc.), a patient computing device 2416 (e.g., phone, computer, tablet, etc.), and/or patient artificial intelligent devices 2417 (e.g., Amazon® Alexa, Google® Assistant).

FIG. 24 illustrates a medical device as an ambulatory medical device 2415. Examples of ambulatory devices include wearable or implantable neuromodulators. The external system 2414 can include a network of computers, including computer(s) remotely located from the ambulatory medical device 2415 that are capable of communicating via one or more communication networks with the programmer 2411 and/or the remote control. The remotely located computer(s) and the ambulatory medical device 2415 can be configured to communicate with each other via another external device such as the programmer 2411 or the remote control. The remote-control device and/or the programmer 2411 can allow a user (e.g., patient and/or clinician or device rep) to answer questions as part of the data collection process. The ambulatory medical device may include internal or external sensors that can be used to collect data, which can form at least part of the overall pain data, healthcare-related data, or other patient data to be transferred to a data receiving system. Parameter data for the programmed therapy can form at least part of the healthcare-related data to be transferred to a data receiving system.

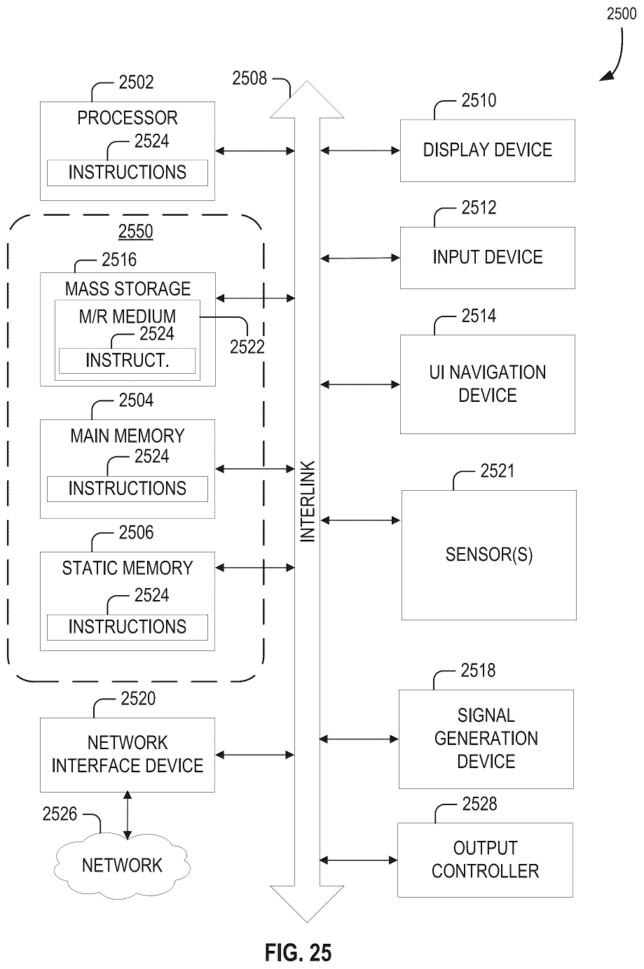

FIG. 25 is a block diagram illustrating a machine in the example form of a computer system 2500, within which a set or sequence of instructions can be executed to cause the machine to perform any one of the methodologies discussed herein, according to an example embodiment. In alternative embodiments, the machine operates as a standalone device or can be connected (e.g., networked) to other machines.

In a networked deployment, the machine may operate in the capacity of either a server or a client machine in server-client network environments, or it may function as a peer machine in peer-to-peer (or distributed) network environments. The machine can be a personal computer (PC), a tablet PC, a hybrid tablet, a personal digital assistant (PDA), a mobile telephone, an implantable pulse generator (IPG), an external remote control (RC), a User's Programmer (UP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 2500 includes at least one processor 2502 (e.g., a central processing unit (CPU), a graphics processing unit (GPU) or both, processor cores, compute nodes, etc.), a main memory 2504 and a static memory 2506, which communicate with each other via a link 2508 (e.g., bus). The computer system 2500 can further include a video display unit 2510, an alphanumeric input device 2512 (e.g., a keyboard), and a user interface (UI) navigation device 2514 (e.g., a mouse). In one embodiment, the video display unit 2510, input device 2512 and UI navigation device 2514 are incorporated into a touch screen display. The computer system 2500 can additionally include a storage device 2516 (e.g., a drive unit), a signal generation device 2518 (e.g., a speaker), an output controller 2528, a network interface device 2520, and one or more sensors 2521, such as a global positioning system (GPS) sensor, compass, accelerometer, or another sensor. It will be understood that other forms of machines or apparatuses (such as PIG, RC, CP devices, and the like) that are capable of implementing the methodologies discussed in this disclosure may not incorporate or utilize every component depicted in FIG. 25 (such as a GPU, video display unit, keyboard, etc.).

The storage device 2516 includes a machine-readable medium 2522 on which is stored one or more sets of data structures and instructions 2524 (e.g., software) embodying or utilized by any one or more of the methodologies or functions described herein. The instructions 2524 can also reside, completely or at least partially, within the main memory 2504, static memory 2506, and/or within the processor 2502 during execution thereof by the computer system 2500, with the main memory 2504, static memory 2506, and the processor 2502 also constituting machine-readable media.

While the machine-readable medium 2522 is illustrated in an example embodiment to be a single medium, the term “machine-readable medium” can include a single medium or multiple media (e.g., a centralized or distributed database, and/or associated caches and servers) that store the one or more instructions 2524. The term “machine-readable medium” shall also be taken to include any tangible (e.g., non-transitory) medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 2524 can further be transmitted or received over a communications network 2526 using a transmission medium via the network interface device 2520 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

As used herein, the terms “machine-storage medium,” “device-storage medium,” “machine-readable medium,” and “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable read-only memory (EEPROM), field-programmable gate arrays (FPGAs), and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions for execution by the machine 2500, and include digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a manner as to encode information in the signal. The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
using at least one medical device to treat a condition by delivering a therapy to a patient;
receiving, from a user via a user interface, criteria related to one or more goals to accomplish while receiving the therapy to treat the condition;
translating, by at least one hardware processor, the criteria related to the one or more goals to a state-based classification defining a plurality of patient states defined by a plurality of parameters;
receiving a first set of parameter values;
identifying a first patient state from among the plurality of patient states using the first set of parameter values; and
generating an output including the first patient state.

2. The method of claim 1, wherein the plurality of parameters includes at least one physiological parameter.

3. The method of claim 1, further comprising:
receiving, from the patient via the user interface, at least one of a patient-supplied ranking of each patient state among the plurality of patient states, a patient-supplied ranking of well-being, or a patient-supplied ranking of each parameter.

4. The method of claim 1, wherein the criteria include at least one of patient-defined criteria, clinician-defined criteria, application-defined criteria, or a combination thereof.

5. The method of claim 1, further comprising:
receiving a second set of parameter values;
identifying a second patient state from among the plurality of patient states using the second set of parameter values; and
generating a comparison state set based on the first set of parameter values and the second set of parameter values.

6. The method of claim 1, wherein the plurality of patient states includes a representation of overall patient health representing a combination of parameter sets including at least one of:
a pain parameter, the pain parameter related to pain of the patient including subjective information provided, via the user interface, by the patient;
a medication parameter;
an activities of daily living parameter;
a mood parameter;
a sleep parameter;
an alertness parameter; and
a mobility parameter.

7. The method of claim 1, further comprising:
determining a multi-step pathway between the plurality of patient states based at least in part on the criteria related to the one or more goals to accomplish while receiving the therapy.

8. The method of claim 7, wherein the multi-step pathway between the plurality of patient states is a non-sequential pathway from the first patient state to a last patient pain state, the last patient pain state being an optimal patient state.

9. The method of claim 1, further comprising:
identifying a time period maintained in the first patient state; and
calculating a dwell time percentage based on the time period maintained among the plurality of patient states.

10. The method of claim 9, further comprising:
mapping the time period maintained in the plurality of patient states to the criteria related to the one or more goals to accomplish while receiving the therapy;
generating a recommendations plan for the therapy; and
providing the recommendations plan for planning treatment decisions.

11. The method of claim 10, further comprising:
identifying one or more confounding variables among the plurality of parameters, the one or more confounding variables being identified as problematic to success of the recommendations plan for the therapy.

12. The method of claim 10, wherein the recommendations plan for the therapy is a closed-loop recommendations plan.

13. A machine-storage medium embodying instructions that, when executed by a machine, cause the machine to perform operations comprising:
using at least one medical device to treat a condition by delivering a therapy to a patient;
receiving, from a user via a user interface, criteria related to one or more goals to accomplish while receiving the therapy to treat the condition;
translating, by at least one hardware processor, the criteria related to the one or more goals to a state-based classification defining a plurality of patient states defined by a plurality of parameters;
receiving a first set of parameter values;
identifying a first patient state from among the plurality of patient states using the first set of parameter values; and
generating an output including the first patient state.

14. The machine-storage medium of claim 13, wherein the plurality of parameters includes at least one physiological parameter.

15. The machine-storage medium of claim 13, further comprising:
receiving, from the patient via the user interface, at least one of a patient-supplied ranking of each patient state among the plurality of patient states, a patient-supplied ranking of well-being, or a patient-supplied ranking of each parameter.

16. The machine-storage medium of claim 15, further comprising:
receiving a second set of parameter values;
identifying a second patient state from among the plurality of patient states using the second set of parameter values; and
generating a comparison state set based on the first set of parameter values and the second set of parameter values.

17. The machine-storage medium of claim 13, wherein the plurality of patient states includes a representation of overall patient health representing a combination of parameter sets including at least one of:
a pain parameter, the pain parameter related to pain of the patient including subjective information provided, via the user interface, by the patient;
a medication parameter;
an activities of daily living parameter;
a mood parameter;
a sleep parameter;
an alertness parameter; and
a mobility parameter.

18. The machine-storage medium of claim 13, further comprising:
determining a multi-step pathway between the plurality of patient states based at least in part on the criteria related to the one or more goals to accomplish while receiving the therapy.

19. The machine-storage medium of claim 18, further comprising:
mapping a time period maintained in the plurality of patient states to the criteria related to the one or more goals to accomplish while receiving the therapy;
generating a recommendations plan for the therapy; and
providing the recommendations plan for planning treatment decisions.

20. A system for managing pain of a patient, the system comprising:
one or more processors; and
one or more memory storing instructions, which when executed by the one or more processors, cause the one or more processors to perform operations that:
receive, from a user via a user interface, criteria related to one or more goals to accomplish while receiving therapy to treat a condition;
translate, by at least one hardware processor, the criteria related to the one or more goals to a state-based classification defining a plurality of patient states defined by a plurality of parameters;
receive a first set of parameter values;
identify a first patient state from among the plurality of patient states using the first set of parameter values; and
generate an output including the first patient state.
